(19) United States
(12) Patent Application Publication
(10) Pub. No.: US 2025/0041336 A1
(43) Pub. Date: Feb. 6, 2025

(54) METHODS AND COMPOSITIONS FOR TREATING CANCER WITH ENGINEERED CELLS

(71) Applicant: Vita Therapeutics, Inc., Baltimore, MD (US)

(72) Inventors: Aihong ZHANG, Baltimore, MD (US); Douglas FALK, Baltimore, MD (US)

(21) Appl. No.: 18/696,325

(22) PCT Filed: Sep. 28, 2022
(86) PCT No.: PCT/US2022/077181

§ 371 Date: Mar. 27, 2024

Related U.S. Application Data
(60) Provisional application No. 63/250,095, filed on Sep. 29, 2021.

(51) Int. Cl. A61K 35/17; A61P 35/00; C07K 14/54; C07K 14/705; C07K 14/725; C12N 5/0789

(52) U.S. Cl. CPC A61K 35/17; A61P 35/00; C07K 14/5434; C07K 14/7051; C07K 14/70521; C07K 14/70575; C12N 5/0647; C07K 2319/03; C12N 2510/00

(57) ABSTRACT

The present disclosure provides new anti-cancer immune cells engineered to express chimeric receptors which, unlike the conventional chimeric antigen receptors (CAR), employ the extracellular domain of a receptor that binds a ligand which may be expressed on a target tumor cell. The immune cell is preferably an immature myeloid cell that is p50 deficient. Such an engineered immune cell exhibits improved therapeutic efficacy as compared to the conventional immune cell therapies and is more broadly applicable to different types of cancers.

### CROSS REFERENCE TO RELATED APPLICATIONS

This application claims the benefit under 35 U.S.C. § 119(e) of the U.S. Provisional Application Ser. No. 63/250,095, filed Sep. 29, 2021, the content of which is hereby incorporated by reference in its entirety.

### REFERENCE TO AN ELECTRONIC SEQUENCE LISTING

The contents of the electronic sequence listing (341203.xml; Size: 50,207 bytes; and Date of Creation: Sep. 28, 2022) is herein incorporated by reference in its entirety.

### BACKGROUND

Chimeric antigen receptor T cells (also known as CAR T cells) are T cells that have been genetically engineered to produce an artificial T cell receptor for use in immunotherapy. CAR-T therapy has the potential to improve the management of lymphomas and possibly solid cancers. A number of CAR-T products have been approved by the FDA for the treatment of cancer, including those targeting CD19 or the B-cell maturation antigen (BCMA).

There are serious side effects, however, that result from CAR-T cells being introduced into the body, including cytokine release syndrome and neurological toxicity. There are also concerns about long-term patient survival, as well as pregnancy complications in female patients.

There is also the unlikely possibility that the engineered CAR-T cells will themselves become transformed into cancerous cells through insertional mutagenesis, due to the viral vector inserting the CAR gene into a tumor suppressor or oncogene in the host T cell's genome.

In addition, the efficacy of the current CAR-T therapies for treating solid cancer is limited by several factors, including the inherent heterogeneity of the cancer cells, strong immunosuppressive tumor microenvironment (TME) contributed by myeloid-derived suppressor cells (MDSC) and/or tumor promoting M2 macrophages, and the lack of tumor penetration by the adoptive transferred immune cells.

### SUMMARY

The present disclosure provides new anti-cancer immune cell therapy approaches with improved therapeutic efficacy as compared to the conventional immune cell therapies. In particular, the present technology enables more effective penetration of solid tumors and is more broadly applicable to various different types of cancers. It is contemplated that these added benefits are at least in part due to the new immune cell therapies' ability to modulate and reverse the immune suppressive TME, to enhance phagocytosis of tumor cells, and to increase activation of tumor-specific cytotoxic T cells.

In accordance with one embodiment of the present disclosure, therefore, provided is a chimeric receptor comprising, from the N-terminus to the C-terminus, an extracellular domain of a receptor to a tumor-associated ligand, a transmembrane domain, and a CD3 intracellular domain.

In some embodiments, the receptor and the ligand have a binding affinity that is between 100 μM and 100 nM (EC₅₀). In some embodiments, the extracellular domain comprises a ligand-binding domain. In some embodiments, the receptor is selected from the group consisting of PD1, SIRPα, Siglec-10, CTLA-4, CXCR-4, CCR-2, CXCR2, CCR7, CD80, TIM-3, LAG3 and TREM2. In some embodiments, the receptor is PD1.

In some embodiments, the chimeric receptor further includes a costimulatory domain. In some embodiments, the costimulatory domain is a signaling domain of a protein selected from the group consisting of CD28, CD27, OX40, CD40, CD80, CD86, and 4-1BB. In some embodiments, the chimeric receptor does not include a costimulatory domain.

Also provided, in one embodiment, is an immune cell that comprises the chimeric receptor of the present disclosure. In some embodiments, the immune cell is selected from the group consisting of myeloid cell, natural killer (NK) cell, T cell, tumor infiltrating lymphocyte, natural killer T (NKT) cell.

In some embodiments, the immune cell is deficient of the p50 subunit encoded by the nfkb1 gene, such as an immune cell that does not express a p50 subunit or has reduced levels of p50. In some embodiments, the immune cell is a myeloid cell. In some embodiments, the immune cell is a p50 deficient immature myeloid cell.

In some embodiments, the immune cell further comprises exogenous polynucleotide encoding a proinflammatory cytokine. In some embodiments, the proinflammatory cytokine is selected from the group consisting of IL-12, IFN-γ, TNF-α, and IL-1β. In some embodiments, the immune cell further comprises a kill switch. In some embodiments, the kill switch is selected from the group consisting of HSV-TK, truncated EGFR (tEGFR), CD19, and CD20.

Also provided, in one embodiment, is a polynucleotide encoding the chimeric receptor of the present disclosure. In some embodiments, the polynucleotide further encodes a proinflammatory cytokine. In some embodiments, the polynucleotide further encodes a kill switch.

Methods for using the compositions of the present disclosure, e.g., chimeric receptors and immune cells, to treat cancer are also provided. In some embodiments, the cancer is a solid tumor, a leukemia or lymphoma. In some embodiments, the cancer is a solid tumor. In some embodiments, the cancer is selected from the group consisting of non-small cell lung cancer (NSCLC), small cell lung cancer, prostate cancer, pancreatic ductal carcinoma, neuroblastoma, glioblastoma, ovarian cancer, melanoma, and breast cancer. In some embodiments, the cancer is metastatic.

### BRIEF DESCRIPTION OF THE DRAWINGS

FIG. 1 shows the organization of an illustrative vector that encodes a chimeric receptor and other useful components of an engineered immune cell of the present technology. The vector encodes a preprotein that includes a leader peptide from EF1α, a chimeric receptor that includes an extracellular domain of PD1, a CD8 hinge region, a CD8 transmembrane domain, a CD40 costimulatory domain, the CD3 activation domain, a protease digestion site P2A, a kill switch (truncated EGFR (tEGFR)), a protease digestion site T2A, and an IL-12 (including p40 and p35, connected through a short G₆S linker (SEQ ID NO:36) linker) as the additional proinflammatory cytokine. Upon protease treatments, the preprotein can be split into the chimeric receptor, the tEGFR kill switch, and the IL-12 cytokine. LTR: Long Terminal Repeats, EF1a: Elongation Factor 1a promoter, CARIR: CAR-Like Immune Receptor, P2A and T2A: Self cleaving peptide sequences, tEGFR: Truncated Epidermal Growth Factor Receptor, IL-12: recombinant proinflammatory cytokine IL-12, PD1: extracellular domain of PD-1 receptor, CD40: CD40 co-stimulatory domain, CD3ζ: CD3 zeta cytoplasmic domain. P40 and p35: p40 and p35 subunits of IL-12.

FIG. 2A-C show lentiviral mediated CARIR expression in human monocytic THP-1 cells. A. Diagrams for configuration of PD-1 CARIR vectors varying in the intracellular signaling domain(s) and with or without the cytokine IL-12 co-expression. B. lentiviral mediated CARIR transduction in human monocytic THP-1 cells with decreasing multiplicity of infection (MOI). CARIR expression was measured by flow cytometry through detecting surface expression of PD-1. C. Lentiviral mediated CARIR expression in human monocytic THP-1 cells at MOI of 10.

FIG. 3A-C show CARIR expression in transduced human monocytic THP-1 cells and its binding with human PD-L1 ligand. A. Diagram for a CARIR-z lentiviral vector that encoding a co-expressed Neon green marker. B. Co-expression of PD-1 and Neon green marker in transduced human monocytic THP-1 cells, as measured by flow cytometry. C. PD-1 CARIR expressed on transduced THP-1 cells binds biotinylated human PD-L1. CARIR transduced THP-1 cells were stained without or with increasing amount of biotinylated human PD-L1, followed by staining with streptavidin APC.

FIG. 4A-B show functional expression of CARIR in human monocytic THP-1 cells. A. Human PD-L1 expression in wild type RM-1 cells or RM-1^{hPD-L}. The latter was engineered to over-express human PD-L1 through lentiviral mediated transduction. B. Upregulation of CD86 and to a much lesser degree, CD80, in CARIR transduced THP-1 cells after co-culture with RM-1 cells that engineered to express human PD-L1. THP-1 cells were either left not transduced (UTD) or transduced with human CARIR that lacks intracellular CD3 zeta chain (hCARIR-Δz), human CARIR (hCARIR-z), or a mouse analogue of hCARIR-z (mCARIR-z). The THP-1 cells were then stimulated with 1 ng/ml PMA for 24 hours, followed by the indicated co-culture treatment for 3 days. The cells were analyzed by flow cytometry for CD80 and CD86 expression. The cells were gated on THP-1 cells based on FSC/SSC parameter.

FIG. 5A-C show CARIR-mediated enhancement of target cell phagocytosis by transduced human monocytic THP-1 cells. A. Procedures for the phagocytosis experiment. The effector cells were prepared from THP-1 cells that were not transduced (THP-1), or lentivirally transduced to express a CARIR that lacks cytosolic domain of CD3 zeta (CARIR-Δz THP-1) or a CARIR that contains an intact cytosolic domain of CD3 zeta (CARIR-z THP-1). The target cells were either wild type RM-1 cells (RM-1) or the RM-1 cells that were lentivirally transduced to overexpress human PD-L1 (RM-1^{hPD-L1}). The ratio of effector and target cells was at 5:1 during the co-culture. B. Dot plots showing phagocytosis events in the Q2 quadrant, which is CellTrace CFSE and violet double positive. The cells were gated on FSC/SSC, singlets, and viable cells. C. Bar graph showing that CARIR expression significantly enhanced the phagocytosis of the target cells. The enhancement was dependent on the interaction between human PD-L1 on the target cells and PD-1 CARIR expressed on the effector cells, and the subsequent cell signaling. The cells were gated on FSC/SSC, singlets, viable cells, and CellTrace Violet⁺ effector THP-1 cells. The data were expressed as mean±SEM. ****p<0.0001, one-way ANOVA compared to the CARIR-z THP-1+RM-1hPD-L1 group.

FIG. 6 shows efficient CRISPR/Cas9 mediated NFκB-1 (p50) gene knockout in human and mouse HSCs. Human CD34+ hematopoietic stem cells (HSCs) isolated from mobilized peripheral blood from different donors were electroporated with ribonucleoprotein (RNP) complex containing 2 guide RNAs. The % indel was evaluated by TIDE analysis. Data are expressed as mean±SEM.

FIG. 7A-D show infusion of mouse immature myeloid cells (IMC) that express a murine analogue of CARIR-z (CARIR-IMC) or p50^{-/-}-IMC slows 4T1 tumor growth and prolongs survival in syngeneic mouse model. A. Schematic timeline for the animal experiment. Syngeneic Balb/c mice were subcutaneously implanted with 5×10⁴ 4T1 breast cancer cells on day −7. Starting on day 0, the mice were treated with 3 weekly dose of IMC (10×10⁶ for each dose) or PBS as indicated. B. Tumor volume measurements. C. Probability of survival. D. Body weight over the course of the experiment. Data are presented as mean±SEM. *p<0.01: CARIR-IMC vs WT-IMC or PBS, p50^{-/-}-IMC vs WT-IMC or PBS, One-way ANOVA. #p<0.05: CARIR-IMC vs PBS, p<0.01: CARIR-IMC vs WT-IMC, p50^{-/-}-IMC vs WT-IMC or PBS, Gehan-Breslow-Wilcoxon test.

FIG. 8 shows NFκB-1 (p50) knock out in human hematopoietic stem cells (HSCs) does not affect CARIR-z expression following lentiviral transduction. HSCs^{p50KO} was generated by CRISPR/Cas9 mediated NFκB-1 (p50) gene knockout in human CD34+ HSCs that isolated from mobilized peripheral blood. Non-modified human HSCs or HSCs^{p50KO} were transduced with lentiviral vector encoding CARIR-z at the MOI of 10. Five days following transduction, CARIR expression was evaluated by flow cytometry based on PD-1 expression.

FIG. 9A-C show CARIR expression in transduced human primary T cells, NK cells, and NKT cells. Human PBMC isolated from healthy donor were stimulated with anti-CD3/CD28 for 24 hrs in the presence of 200 U/ml human recombinant IL-2. The activated cells were then transduced with CARIR-z lentiviral vector at MOI of 25 through spinoculation. Three days following the spinoculation, the percentage of CARIR expression among T cells (CD3+), NK cells (CD3-CD56+), and NKT cells (CD3+CD56+) was analyzed based on PD-1 expression by flow cytometry. A. CARIR expression in CD3+ T cells. B. CARIR expression in CD3-CD56+NK cells. C. CARIR expression in CD3+CD56+ NKT cells.

FIG. 10A-F show that CARIR modified human macrophages efficiently phagocytosed target cells that engineered to express human PD-L1. For generating CARIR modified human macrophages to serve as the effector cells, human CD34+ hematopoietic stem cells (HSCs) were engineered to express CARIR through lentiviral transduction, and then differentiated into macrophages. For target cells, both wild type RM1 cells and RM1^{hPD-L1} cells were used. The later line was established by overexpressing human PD-L1 through lentiviral vector-mediated transduction. A. A flow chart for the experimental procedures. B. The flow data showing the % phagocytosis of human CARIR-z macrophage on wild RM1 or RM1^{hPD-L1} target cells. C. The flow data showing the % phagocytosis of human CARIR-40z macrophage on wild RM1 or RM1^{hPD-L1} target cells. D. The flow data showing the % phagocytosis of human CARIR-40 macrophage on wild RM1 or RM1^{hPD-L1} target cells. E. The flow data showing the % phagocytosis of human CARIR-z-12 macrophage on wild RM1 or RM1^{hPD-L1} target cells. F. The bar graph summarizes the flow data shown in panels from B to E. The cells were gated on CellTrace Violet⁺ macrophages. The CellTrace Violet and CFSE double positive population represent macrophages that have phagocytosed target cells. **p<0.01, ***p<0.001, and ****p<0.0001 by unpaired student t test analyzed using Prism software.

FIG. 11A-D show that CARIR expression enhanced the phagocytosis on human triple negative breast cancer cells by the engineered human THP-1 macrophages. To serve as the effectors, human monocytic THP-1 cells were engineered to express either CARIR-Δz or CARIR-z through lentiviral transduction, and then differentiated to macrophages by PMA treatment. MDA-MB-231 tumor cells that naturally express PD-L1 were used to serve as the target cells. A. A flow chart for the experimental procedures. B. Histogram showing the PD-L1 expression on MDA-MB-231 cells. C. Representative flow plots showing % phagocytosis on MDA-MB-231 tumor cells. The cells were gated on CellTrace Violet THP-1 macrophages. The CellTrace Violet and CellTrace Yellow double positive population represent macrophages that have phagocytosed target cells. D. Bar graph summarizes the phagocytosis result shown in panel C. ***p<0.001, and ****p<0.0001 by ordinary one-way ANOVA using Prism software.

FIG. 12 illustrates the structure of certain additional CAR-like Immune Receptors (CARIR) with extracellular domain derived from different receptors.

FIG. 13A-B show diagrams for additional CAR-like Immune Receptors (CARIR) with different intracellular domain(s).

FIG. 14 illustrates a process for adoptive engineered-myeloid cell therapy for treating cancer.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

### Chimeric Receptors

In various aspects of the present technology, an immune cell is transduced to express a chimeric receptor that helps target the immune cell to a tumor cell. The chimeric receptor, also referred to as a “CAR-like immune receptor,” or “CARIR”, like a conventional CAR, includes an extracellular targeting domain, a transmembrane domain, and one or more costimulatory domains or signal domains.

A conventional CAR includes an antibody or antigen-binding fragment, such as a single chain fragment (scFv), as the extracellular targeting domain to bind to a target molecule, such as a tumor-associated antigen (TAA). By contrast, the chimeric receptors of the present disclosure, in some embodiments, employs the extracellular binding domain of a natural receptor protein that can bind to the target protein, through a conventional ligand-receptor interaction.

For instance, various inhibitory receptors are expressed on immune cells, such as myeloid cells. Non-limiting examples include PD1 which can bind to ligand PD-L1, SIRPα which can bind to CD47, Siglec-10 which can bind to CD52 and CD24, CTLA-4 which can bind to B7-1 and B7-2, TIM-3 which can bind to Gal-9, PtdSer, HMGB1 and CEACAM1, and LAG3 which can bind to MHC class II and FGL1. In some embodiments, the receptor is PD1, SIRPα, Siglec-10, or CTLA-4. In some embodiments, the receptor is CD80 which binds to CD28 and PD-L1 with low affinity. In some embodiments, the receptor is TREM2.

Various chemokine receptors which are expressed on immune cells also include such extracellular domains capable of binding to the corresponding ligands. For instance, the chemokine receptor CXCR4 can bind to SDF-1, ubiquitin and MIF, CCR2 can bind to chemokine CCL2, CXCR2 can bind to CXCL1, and CCR7 can bind to CCL19 and CCL21.

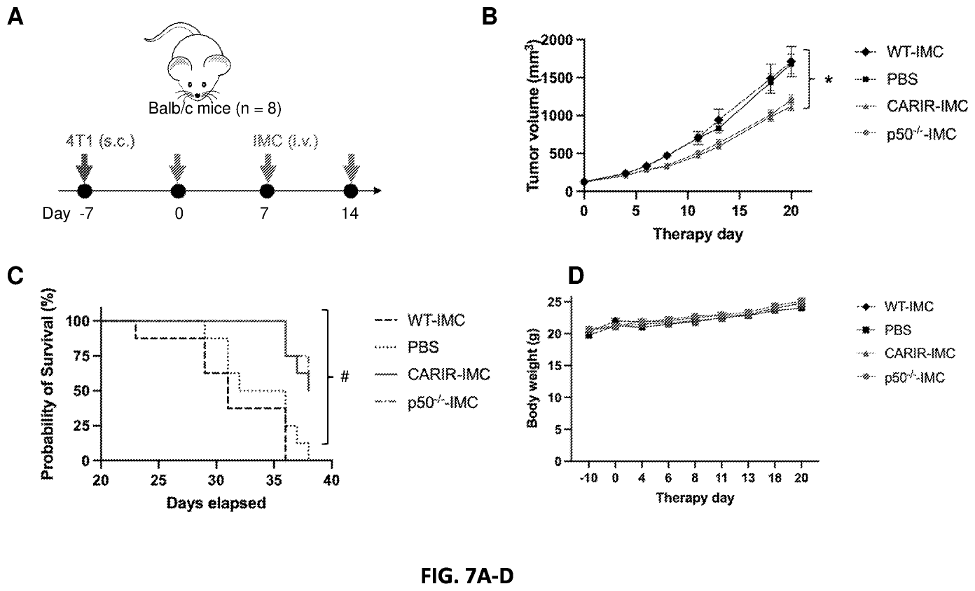

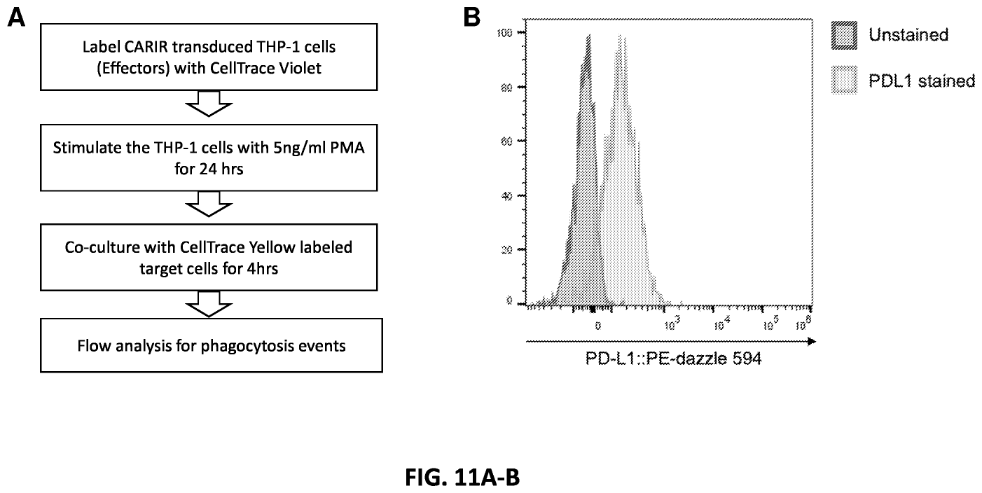

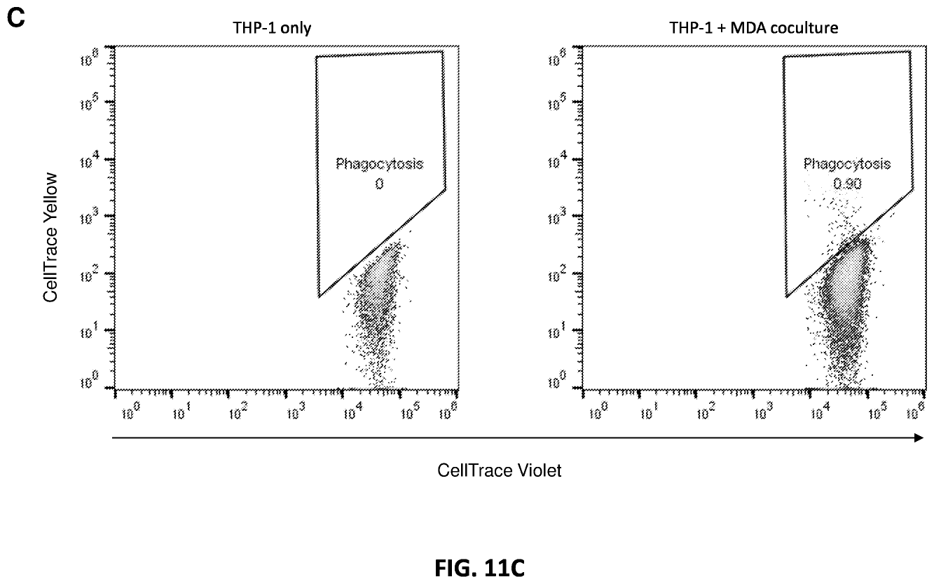

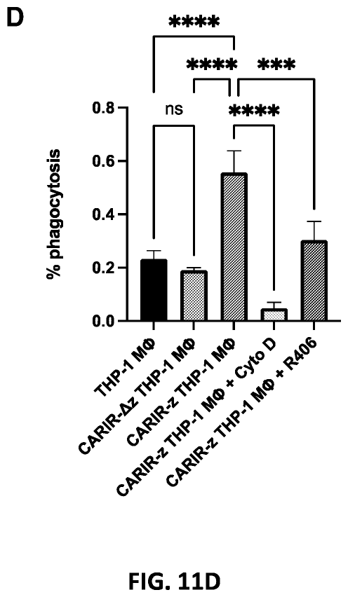

As demonstrated in the accompanying experimental examples, when a CARIR that contained the extracellular domain of PD1 was expressed on an immune cell, such as an immature myeloid cell or a macrophage, the engineered immune cell was able to bind to tumor cells expressing PD-L1. Consequently, such binding initiated phagocytosis (see, e.g., FIG. 11D) or PD-1/PD-L1 signaling leading to inhibition of tumor growth (see, e.g., FIG. 7B-C).

Such anti-tumor effects of the CARIR molecules, however, were unexpected. It is commonly known that therapeutic antibodies typically have a binding affinity on the scale of 0.1-10 nM (EC₅₀). For instance, the EC₅₀ of anti-PD1 antibodies pembrolizumab and nivolumab are 2.440 nM and 5.697 nM, respectively. The affinity between the natural ligands and receptors, however, can be considerably lower. For example, the EC₅₀ between PD-1 and PD-L1 is 7 μM and that between SIRPα and CD47 is 2 μM, both of which are about 1000 times weaker than antibodies. With a 1000-fold lower binding affinity but achieving in vivo anti-tumor effects in a model which is known for its poor response to traditional anti-PD1/PD-L1 antibody blockade therapy (FIG. 7B-C), the instantly disclosed CARIR truly have exhibited unexpected results.

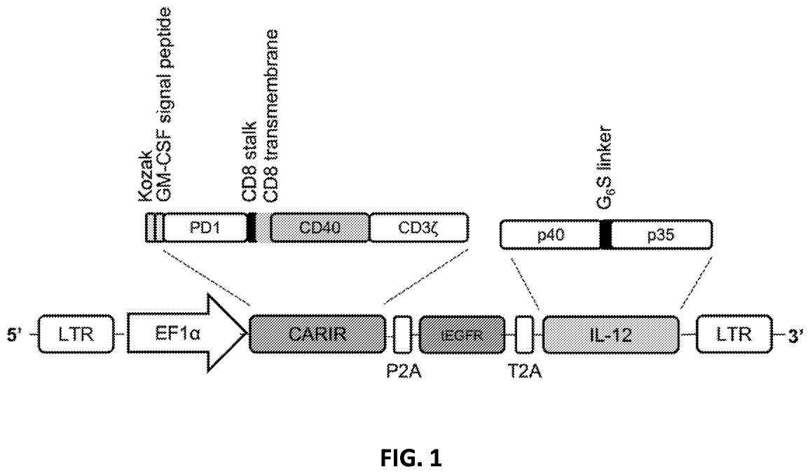

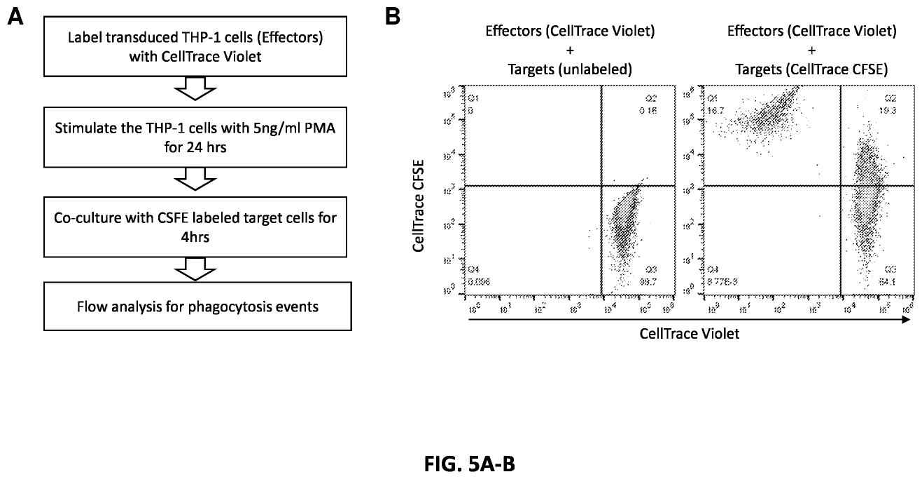

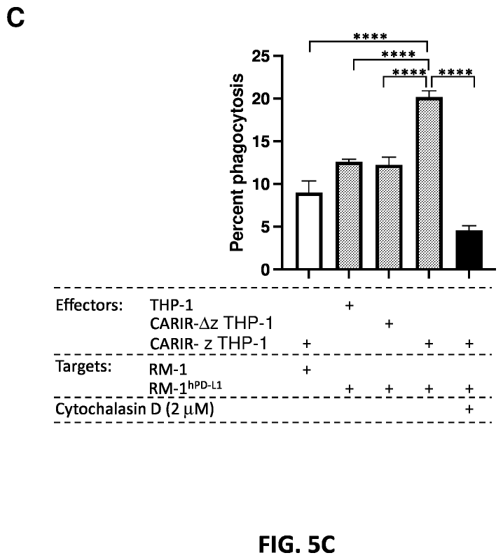

Another interesting discovery is that, unlike a conventional CAR, a CARIR does not necessarily require an intracellular costimulatory domain to be fully functional. For instance, one of the best-performing CARIR molecules in the Examples is CARIR-z (see, e.g., FIG. 5C), which contained the CD3 intracellular domain but not the CD40 costimulatory domain (see structures in FIG. 1).

The full-length sequences of these example receptors are provided in Table 1, along with their extracellular targeting domains. For each receptor, a core extracellular domain (ECD) is provided, which shows the minimum sequence required for binding to the ligand. Sometimes, an extended ECD sequence is also provided, which is slightly longer than the core ECD sequence.

TABLE 1
Extracellular Domains of Example Receptors

| Receptor | Sequences |
|---|---|
| PD1 | Full length: (SEQ ID NO: 1) |
| | MQIPQAPWPVVWAVLQLGWRPGWFLDSPDRPWNPPTFSPALLVVTEGDNATFTCSFSN |
| | TSESFVLNWYRMSPSNQTDKLAAFPEDRSQPGQDCRFRVTQLPNGRDFHMSVVRARRN |
| | DSGTYLCGAISLAPKAQIKESLRAELRVTERRAEVPTAHPSPSPRPAGQFQTLVVGVV |
| | GGLLGSLVLLVWVLAVICSRAARGTIGARRTGQPLKEDPSAVPVFSVDYGELDFQWRE |
| | KTPEPPVPCVPEQTEYATIVFPSGMGTSSPARRGSADGPRSAQPLRPEDGHCSWPL |
| | Core ECD binding sequence: (SEQ ID NO: 2) |
| | MSPSNQTDKLAAFPEDRSQPGQDCRFRVTQ |
| | Extended ECD binding sequence: (SEQ ID NO: 3) |
| | MSPSNQTDKLAAFPEDRSQPGQDCRFRVTQLPNGRDFHMSVVRARRNDSGTYLCGAIS |
| | LAPKAQIKESLRAELRVTERRAEVPTAHPSPSPRPAGQFQTLV |
| SIRPα | Full length: (SEQ ID NO: 4) |
| | MEPAGPAPGRLGPLLCLLLAASCAWSGVAGEEELQVIQPDKSVLVAAGETATLRCTAT |
| | SLIPVGPIQWFRGAGPGRELIYNQKEGHFPRVTTVSDLTKRNNMDFSIRIGNITPADA |
| | GTYYCVKFRKGSPDDVEFKSGAGTELSVRAKPSAPVVSGPAARATPQHTVSFTCESHG |
| | FSPRDITLKWFKNGNELSDFQTNVDPVGESVSYSIHSTAKVVLTREDVHSQVICEVAH |
| | VTLQGDPLRGTANLSETIRVPPTLEVTQQPVRAENQVNVTCQVRKFYPORLQLTWLEN |
| | GNVSRTETASTVTENKDGTYNWMSWLLVNVSAHRDDVKLTCQVEHDGQPAVSKSHDLK |
| | VSAHPKEQGSNTAAENTGSNERNIYIVVGVVCTLLVALLMAALYLVRIRQKKAQGSTS |
| | STRLHEPEKNAREITQDTNDITYADLNLPKGKKPAPQAAEPNNHTEYASIQTSPQPAS |
| | EDTLTYADLDMVHLNRTPKQPAPKPEPSFSEYASVQVPRK |
| | Core ECD binding sequence: (SEQ ID NO: 5) |
| | EEELQVIQPDKSVLVAAGETATLRCTATSLIPVGPIQWFRGAGPGRELIYNQKEGHFP |
| | RVTTVSDLTKRNNMDFSIRIGNITPADAGTYYCVKFRKGSPDDVE |
| | Extended ECD binding sequence: (SEQ ID NO: 6) |
| | EEELQVIQPDKSVLVAAGETATLRCTATSLIPVGPIQWFRGAGPGRELIYNQKEGHFP |
| | RVTTVSDLTKRNNMDFSIRIGNITPADAGTYYCVKFRKGSPDDVEFKSGAGTELSVRA |
| | KPSAPVVSGPAARATPQHTVSFTCESHGFSPRDITLKWFKNGNELSDFQTNVDPVGES |
| | VSYSIHSTAKVVLTREDVHSQVICEVAHVTLQGDPLRGTANLSETIRVPPTLEVTQQP |
| | VRA |
| Siglec-10 | Full length: (SEQ ID NO: 7) |
| | MLLPLLLSSLLGGSQAMDGRFWIRVQESVMVPEGLCISVPCSFSYPRQDWTGSTPAYG |
| | YWFKAVTETTKGAPVATNHQSREVEMSTRGRFQLTGDPAKGNCSLVIRDAQMQDESQY |
| | FFRVERGSYVRYNFMNDGFFLKVTALTQKPDVYIPETLEPGQPVTVICVENWAFEECP |
| | PPSFSWTGAALSSQGTKPTTSHFSVLSFTPRPQDHNTDLTCHVDFSRKGVSAQRTVRL |
| | RVAYAPRDLVISISRDNTPALEPQPQGNVPYLEAQKGQFLRLLCAADSQPPATLSWVL |
| | QNRVLSSSHPWGPRPLGLELPGVKAGDSGRYTCRAENRLGSQQRALDLSVQYPPENLR |
| | VMVSQANRTVLENLGNGTSLPVLEGQSLCLVCVTHSSPPARLSWTQRGQVLSPSQPSD |
| | PGVLELPRVQVEHEGEFTCHARHPLGSQHVSLSLSVHYSPKLLGPSCSWEAEGLHCSC |
| | SSQASPAPSLRWWLGEELLEGNSSQDSFEVTPSSAGPWANSSLSLHGGLSSGLRLRCE |
| | AWNVHGAQSGSILQLPDKKGLISTAFSNGAFLGIGITALLFLCLALIIMKILPKRRTQ |
| | TETPRPRFSRHSTILDYINVVPTAGPLAQKRNQKATPNSPRTPLPPGAPSPESKKNQK |
| | KQYQLPSFPEPKSSTQAPESQESQEELHYATLNFPGVRPRPEARMPKGTQADYAEVKF |
| | Q |
| | Core ECD binding sequence: (SEQ ID NO: 8) |
| | MDGRFWIRVQESVMVPEGLCISVPCSFSYPRQDWTGSTPAYGYWFKAVTETTKGAPVA |
| | TNHQSREVEMSTRGRFQLTGDPAKGNCSLVIRDAQMQDESQYFFRVERGSYVRYNFMN |
| | DGFFLKVTALTQK |
| | Extended ECD binding sequence: (SEQ ID NO: 9) |
| | MDGRFWIRVQESVMVPEGLCISVPCSFSYPRQDWTGSTPAYGYWFKAVTETTKGAPVA |
| | TNHQSREVEMSTRGRFQLTGDPAKGNCSLVIRDAQMQDESQYFFRVERGSYVRYNFMN |
| | DGFFLKVTALTQKPDVYIPETLEPGQPVTVICVFNWAFEECPPPSFSWTGAALSSQGT |
| | KPTTSHFSVLSFTPRPQDHNTDLTCHVDFSRKGVSAQRTVRLRVAYAPRDLVISISRD |
| | NTPALE |
| CTLA-4 | Full length: (SEQ ID NO: 10) |
| | MACLGFQRHKAQLNLATRTWPCTLLFFLLFIPVFCKAMHVAQPAVVLASSRGIASFVC |
| | EYASPGKATEVRVTVLRQADSQVTEVCAATYMMGNELTFLDDSICTGTSSGNQVNLTI |
| | QGLRAMDTGLYICKVELMYPPPYYLGIGNGTQIYVIDPEPCPDSDELLWILAAVSSGL |
| | FFYSFLLTAVSLSKMLKKRSPLTTGVYVKMPPTEPECEKQFQPYFIPIN |
| | Core ECD binding sequence: (SEQ ID NO: 11) |
| | RGIASFVCEYASPGKATEVRVTVLRQADSQVTEVCAATYMMGNELTFLDDSICTGTSS |
| | GNQVNLTIQGLRAMDTGLYICKVELMYPPPYYLGIGNGTQI |
| TIM-3 | Full length: (SEQ ID NO: 12) |
| (havcr2) | MFSHLPFDCVLLLLLLLLTRSSEVEYRAEVGQNAYLPCFYTPAAPGNLVPVCWGKGAC |
| | PVFECGNVVLRTDERDVNYWTSRYWLNGDFRKGDVSLTIENVTLADSGIYCCRIQIPG |
| | IMNDEKFNLKLVIKPAKVTPAPTRQRDFTAAFPRMLTTRGHGPAETQTLGSLPDINLT |
| | QISTLANELRDSRLANDLRDSGATIRIGIYIGAGICAGLALALIFGALIFKWYSHSKE |
| | KIQNLSLISLANLPPSGLANAVAEGIRSEENIYTIEENVYEVEEPNEYYCYVSSRQQP |
| | SQPLGCRFAMP |
| | Core ECD binding sequence: (SEQ ID NO: 13) |
| | EVEYRAEVGQNAYLPCFYTPAAPGNLVPVCWGKGACPVFECGNVVLRTDERDVNYWTS |
| | RYWLNGDFRKGDVSLTIENVTLADSGIYCCRIQIPGIMNDEKFNLKLVIKPAKVTPAP |
| | TRQRDFTAAFPRMLTTRGHGPAETQTLGSLPDINLTQISTLANELRDSRLANDLRDSG |
| | ATIRIG |
| LAG3 | Full length: (SEQ ID NO: 14) |
| | MWEAQFLGLLFLQPLWVAPVKPLQPGAEVPVVWAQEGAPAQLPCSPTIPLQDLSLLRR |
| | AGVTWQHQPDSGPPAAAPGHPLAPGPHPAAPSSWGPRPRRYTVLSVGPGGLRSGRLPL |
| | QPRVQLDERGRQRGDFSLWLRPARRADAGEYRAAVHLRDRALSCRLRLRLGQASMTAS |
| | PPGSLRASDWVILNCSFSRPDRPASVHWFRNRGQGRVPVRESPHHHLAESFLFLPQVS |
| | PMDSGPWGCILTYRDGFNVSIMYNLTVLGLEPPTPLTVYAGAGSRVGLPCRLPAGVGT |
| | RSFLTAKWTPPGGGPDLLVTGDNGDFTLRLEDVSQAQAGTYTCHIHLQEQQLNATVTL |
| | AIITVTPKSFGSPGSLGKLLCEVTPVSGQERFVWSSLDTPSQRSFSGPWLEAQEAQLL |
| | SQPWQCQLYQGERLLGAAVYFTELSSPGAQRSGRAPGALPAGHLLLFLILGVLSLLLL |
| | VTGAFGFHLWRRQWRPRRFSALEQGIHPPQAQSKIEELEQEPEPEPEPEPEPEPEPEP |
| | EQL |
| | Core ECD binding sequence: (SEQ ID NO: 15) |
| | LQPGAEVPVVWAQEGAPAQLPCSPTIPLQDLSLLRRAGVTWQHQPDSGPPAAAPGHPL |
| | APGPHPAAPSSWGPRPRRYTVLSVGPGGLRSGRLPLQPRVQLDERGRQRGDFSLWLRP |
| | ARRADAGEYRAAVHLRDRALSCRLRLRLGQASMTASPPGSLRASDWVILNCSFSRPDR |
| | PASVHWFRNRGQGRVPVRESPHHHLAESFLFLPQVSPMDSGPWGCILTYRDGFNVSIM |
| | YNLTV |
| | Extended ECD binding sequence: (SEQ ID NO: 16) |
| | LQPGAEVPVVWAQEGAPAQLPCSPTIPLQDLSLLRRAGVTWQHQPDSGPPAAAPGHPL |
| | APGPHPAAPSSWGPRPRRYTVLSVGPGGLRSGRLPLQPRVQLDERGRQRGDFSLWLRP |
| | ARRADAGEYRAAVHLRDRALSCRLRLRLGQASMTASPPGSLRASDWVILNCSFSRPDR |
| | PASVHWFRNRGQGRVPVRESPHHHLAESFLFLPQVSPMDSGPWGCILTYRDGFNVSIM |
| | YNLTVLGLEPPTPLTVYAGAGSRVGLPCRLPAGVGTRSFLTAKWTPPGGGPDLLVTGD |
| | NGDFTLRLEDVSQAQAGTYTCHIHLQEQQLNATVTLAIITVTPKSFGSPGSLGKLLCE |
| | VTPVS |
| CXCR4 | Full length: (SEQ ID NO: 17) |
| | MEGISIYTSDNYTEEMGSGDYDSMKEPCFREENANFNKIFLPTIYSIIFLTGIVGNGL |
| | VILVMGYQKKLRSMTDKYRLHLSVADLLFVITLPFWAVDAVANWYFGNFLCKAVHVIY |
| | TVNLYSSVLILAFISLDRYLAIVHATNSQRPRKLLAEKVVYVGVWIPALLLTIPDFIF |
| | ANVSEADDRYICDRFYPNDLWVVVFQFQHIMVGLILPGIVILSCYCIIISKLSHSKGH |
| | QKRKALKTTVILILAFFACWLPYYIGISIDSFILLEIIKQGCEFENTVHKWISITEAL |
| | AFFHCCLNPILYAFLGAKFKTSAQHALTSVSRGSSLKILSKGKRGGHSSVSTESESSS |
| | FHSS |
| | Core ECD binding sequence: (SEQ ID NO: 18) |
| | MEGISIYTSDNYTEEMGSGDYDSMKEPCFREENANFNK |
| CCR2 | Full length: (SEQ ID NO: 19) |
| | MLSTSRSRFIRNTNESGEEVTTFFDYDYGAPCHKFDVKQIGAQLLPPLYSLVFIFGFV |
| | GNMLVVLILINCKKLKCLTDIYLLNLAISDLLFLITLPLWAHSAANEWVFGNAMCKLF |
| | TGLYHIGYFGGIFFIILLTIDRYLAIVHAVFALKARTVTFGVVTSVITWLVAVFASVP |
| | GIIFTKCQKEDSVYVCGPYFPRGWNNFHTIMRNILGLVLPLLIMVICYSGILKTLLRC |
| | RNEKKRHRAVRVIFTIMIVYFLFWTPYNIVILLNTFQEFFGLSNCESTSQLDQATQVT |
| | ETLGMTHCCINPIIYAFVGEKFRRYLSVFFRKHITKRFCKQCPVFYRETVDGVTSTNT |
| | PSTGEQEVSAGL |
| | Core ECD binding sequence: (SEQ ID NO: 20) |
| | LSTSRSRFIRNTNESGEEVTTFFDYDYGAPCHKFDVK |
| CXCR2 | Full length: (SEQ ID NO: 21) |
| | MEDFNMESDSFEDFWKGEDLSNYSYSSTLPPFLLDAAPCEPESLEINKYFVVIIYALV |
| | FLLSLLGNSLVMLVILYSRVGRSVTDVYLLNLALADLLFALTLPIWAASKVNGWIFGT |
| | FLCKVVSLLKEVNFYSGILLLACISVDRYLAIVHATRTLTQKRYLVKFICLSIWGLSL |
| | LLALPVLLFRRTVYSSNVSPACYEDMGNNTANWRMLLRILPQSFGFIVPLLIMLFCYG |
| | FTLRTLFKAHMGQKHRAMRVIFAVVLIFLLCWLPYNLVLLADTLMRTQVIQETCERRN |
| | HIDRALDATEILGILHSCLNPLIYAFIGQKFRHGLLKILAIHGLISKDSLPKDSRPSF |
| | VGSSSGHTSTTL |
| | Core ECD binding sequence: (SEQ ID NO: 22) |
| | EDFNMESDSFEDFWKGEDLSNYSYSSTLPPFLLDAAPCEPESLEINK |
| CCR7 | Full length: (SEQ ID NO: 23) |
| | MDLGKPMKSVLVVALLVIFQVCLCQDEVTDDYIGDNTTVDYTLFESLCSKKDVRNFKA |
| | WFLPIMYSIICFVGLLGNGLVVLTYIYFKRLKTMTDTYLLNLAVADILFLLTLPFWAY |
| | SAAKSWVFGVHFCKLIFAIYKMSFFSGMLLLLCISIDRYVAIVQAVSAHRHRARVLLI |
| | SKLSCVGIWILATVLSIPELLYSDLQRSSSEQAMRCSLITEHVEAFITIQVAQMVIGF |
| | LVPLLAMSFCYLVIIRTLLQARNFERNKAIKVIIAVVVVFIVFQLPYNGVVLAQTVAN |
| | FNITSSTCELSKQLNIAYDVTYSLACVRCCVNPFLYAFIGVKFRNDLFKLFKDLGCLS |
| | QEQLRQWSSCRHIRRSSMSVEAETTTTFSP |
| | Core ECD binding sequence: (SEQ ID NO: 24) |
| | QDEVTDDYIGDNTTVDYTLFESLCSKKDVRNFKA |
| CD80 | Full length: (SEQ ID NO: 41) |
| | MGHTRRQGTSPSKCPYLNFFQLLVLAGLSHFCSGVIHVTKEVKEVATLSCGHNVSVEE |
| | LAQTRIYWQKEKKMVLTMMSGDMNIWPEYKNRTIFDITNNLSIVILALRPSDEGTYEC |
| | VVLKYEKDAFKREHLAEVTLSVKADFPTPSISDFEIPTSNIRRIICSTSGGFPEPHLS |
| | WLENGEELNAINTTVSQDPETELYAVSSKLDFNMTTNHSFMCLIKYGHLRVNQTFNWN |
| | TTKQEHFPDNLLPSWAITLISVNGIFVICCLTYCFAPRCRERRRNERLRRESVRPV |
| | Core ECD binding sequence: (SEQ ID NO: 42) |
| | IFDITNNLSIVIL |
| | Extended ECD binding sequence: (SEQ ID NO: 43) |
| | VIHVTKEVKEVATLSCGHNVSVEELAQTRIYWQKEKKMVLTMMSGDMNIWPEYKNRTI |
| | FDITNNLSIVILALRPSDEGTYECVVLKYEKDAFKREHLAEVTLSVK |

Accordingly, in some embodiments, a chimeric receptor is provided which includes, from the N-terminus to the C-terminus, an extracellular domain of a receptor to a tumor-associated ligand, a transmembrane domain, and a CD3 intracellular domain. In some embodiments, the extracellular domain includes a ligand-binding domain.

In some embodiments, the receptor (or the extracellular domain thereof) has a binding affinity to the ligand. The binding affinity, in some embodiments, is not higher than 0.1 nM (EC₅₀). In some embodiments, the binding affinity is not higher than 1 nM, 5 nM, 10 nM, 50 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, or 100 μM. In some embodiments, the binding affinity is higher than 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 500 nM, 100 nM, 50 nM, 20 nM, or 10 nM. In some embodiments, the binding affinity is between 100 μM and 100 nM, between 100 μM and 200 nM, between 50 μM and 500 nM, or between 20 μM and 1 μM, without limitation.

In some embodiments, the receptor is an inhibitory receptor.

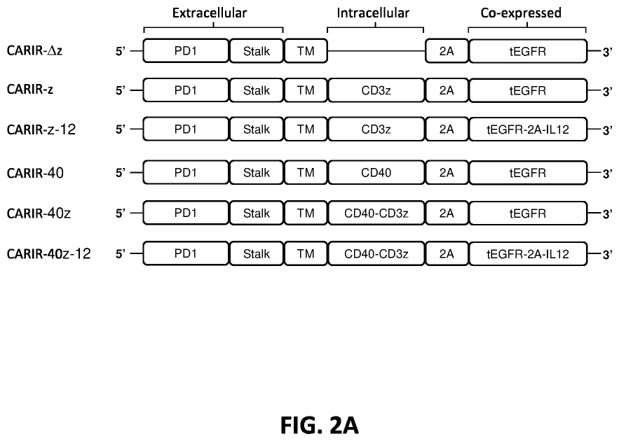

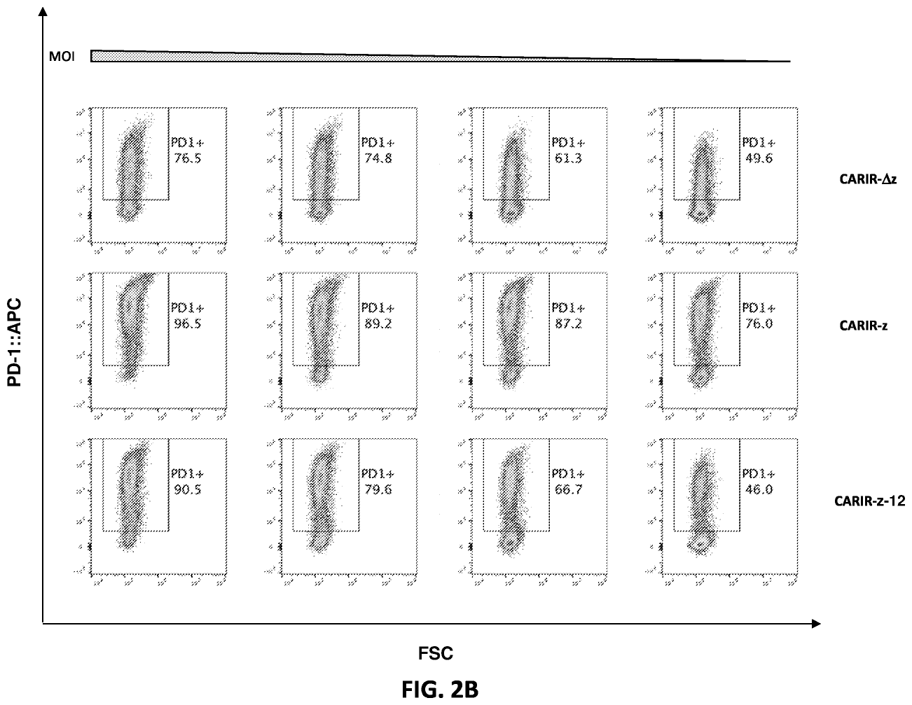

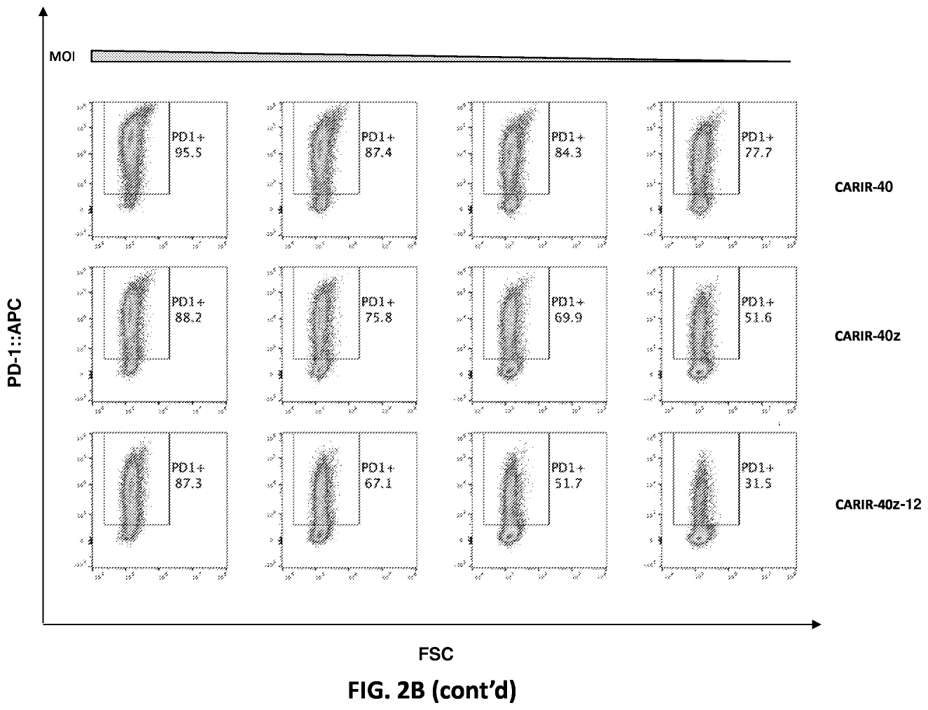

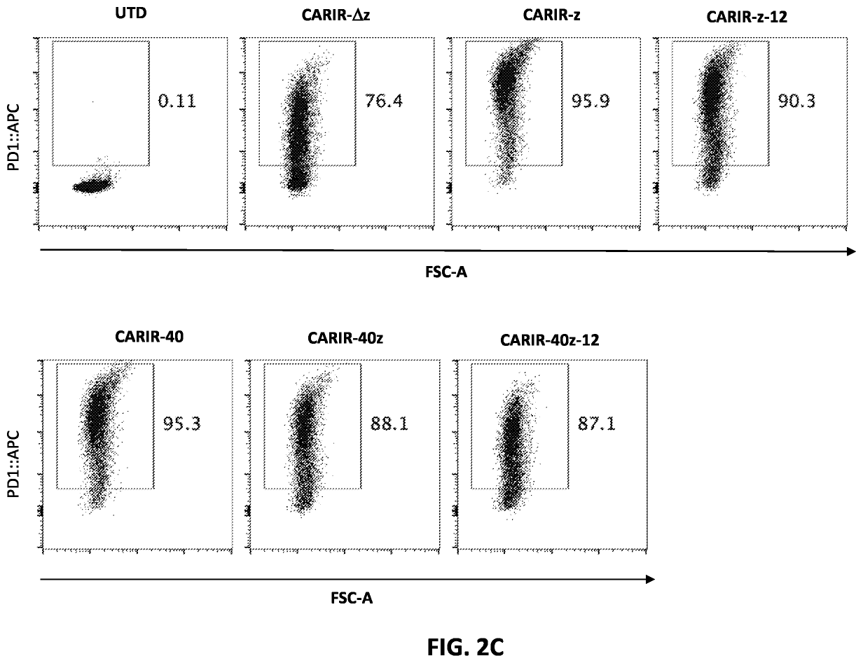

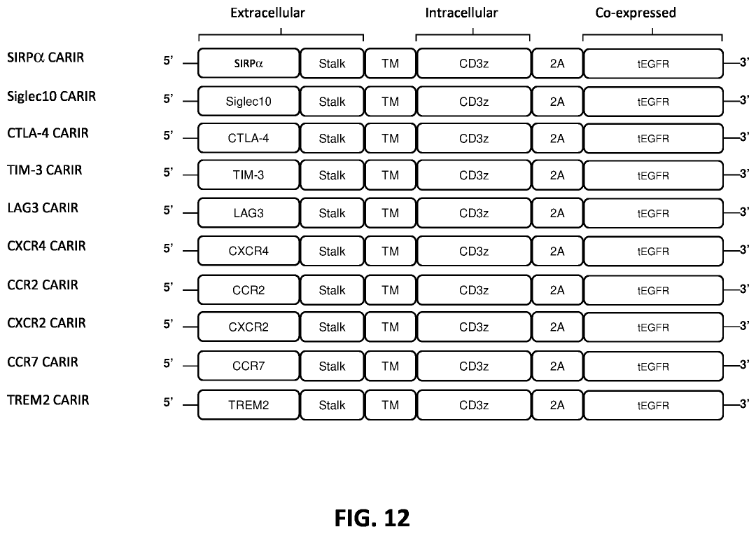

Example receptors include PD1, SIRPα, Siglec-10, CTLA-4, CXCR-4, CCR-2, CXCR2, CCR7, CD80, TIM-3, LAG3 and TREM2 (see, e.g., FIGS. 2A and 12). In a particular embodiment, the receptor is PD1. In another embodiment, the receptor is SIRPα. In another embodiment, the receptor is CD80. Example ligand-binding domains include the sequences provided in SEQ ID NO:2, 5, 8, 11, 13, 15, 18, 20, 24 and 42. Additional examples include SEQ ID NO:3, 6, 9, 16 and 43.

Still, further examples include those that are biologically equivalent to those exemplified above. A biological equivalent to an extracellular binding domain is one that has at least 75%, 80%, 85%, 90%, 95%, 98%, or 99% sequence identity to a reference extracellular binding domain, such as those provided in Table 1. In some embodiments, the substitutions allowed in the designated sequence identities are conservative amino acid substitutions.

The extracellular targeting domain can target the engineered immune cell, which expresses the extracellular targeting domain, to a tumor tissue where the corresponding ligand is expressed. In addition to the extracellular domain, the chimeric receptor also includes other useful elements.

In some embodiments, the chimeric receptor further includes a transmembrane (TM) domain. A transmembrane domain can be designed to be fused to the extracellular domain, optionally through a hinge domain. It can similarly be fused to an intracellular domain, such as a costimulatory domain. In some embodiments, the transmembrane domain can include the natural transmembrane region of a costimulatory domain (e.g., the TM region of a CD28T or 4-IBB employed as a costimulatory domain) or the natural transmembrane domain of a hinge region (e.g., the TM region of a CD8 alpha or CD28T employed as a hinge domain). Example sequences are provided in Table 2.

In some embodiments, the transmembrane domain can be selected or modified by amino acid substitution to avoid binding of such domains to the transmembrane domains of the same or different surface membrane proteins to minimize interactions with other members of the receptor complex. A transmembrane domain can be derived either from a natural or from a synthetic source. When the transmembrane domain is derived from a naturally-occurring source, the domain can be derived from any membrane-bound or transmembrane protein. In some embodiments, a transmembrane domain is derived from CD2, CD3 delta, CD3 epsilon, CD3 gamma, CD4, CD7, CD8a, CD8, CD11a (ITGAL), CD 11b (ITGAM), CD11c (ITGAX), CD11d (ITGAD), CD18 (ITGB2), CD19 (B4), CD27 (TNFRSF7), CD28, CD28T, CD29 (ITGB 1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF 1), CD158A (KTR2DL1), CD158B1 (KIR2DL2), CD158B2 (KIR2DL3), CD158C (KTR3DP1), CD158D (KTRDL4), CD158F 1 (KTR2DL5A), CD158F2 (KTR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (T FRSF18), inducible T cell co-stimulator (ICOS), LFA-1 (CD11a/CD18), KG2C, DAP-10, ICAM-1, Kp80 (KLRF 1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and combinations thereof.

In some embodiments, the transmembrane domain can include a sequence that spans a cell membrane, but extends into the cytoplasm of a cell and/or into the extracellular space. For example, a transmembrane can include a membrane-spanning sequence which itself can further include 1, 2, 3, 4, 5, 6, 7, 8, 9, 10 or more amino acids that extend into the cytoplasm of a cell, and/or the extracellular space. Thus, a transmembrane domain includes a membrane-spanning region, yet can further comprise an amino acid(s) that extend beyond the internal or external surface of the membrane itself; such sequences can still be considered to be a “transmembrane domain.”

In some embodiments, the transmembrane domain of a chimeric receptor of the instant disclosure includes the human CD8a transmembrane domain (SEQ ID NO:28). In some embodiments, the CD8a transmembrane domain is fused to the extracellular domain through a hinge region. In some embodiments, the hinge region includes the human CD8a hinge (SEQ ID NO:27).

In some embodiments, the transmembrane domain is fused to the cytoplasmic domain through a short linker. Optionally, the short peptide or polypeptide linker, preferably between 2 and 10 amino acids in length can form the linkage between the transmembrane domain and a proximal cytoplasmic signaling domain of the chimeric receptor. A glycine-serine doublet (GS), glycine-serine-glycine triplet (GSG), or alanine-alanine-alanine triplet (AAA) provides a suitable linker.

In some embodiments, the chimeric receptor further includes a costimulatory domain. In some embodiments, the costimulatory domain is positioned between the transmembrane domain and an activating domain. Example costimulatory domains include, but are not limited to, CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD18 (ITGB2), CD19 (B4), CD27 (T FRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD 100 (SEMA4D), CD 103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KTR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (TNFRSF 18), inducible T cell co-stimulator (ICOS), LFA-1 (CD 11a/CD 18), KG2C, DAP-10, ICAM-1, Kp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

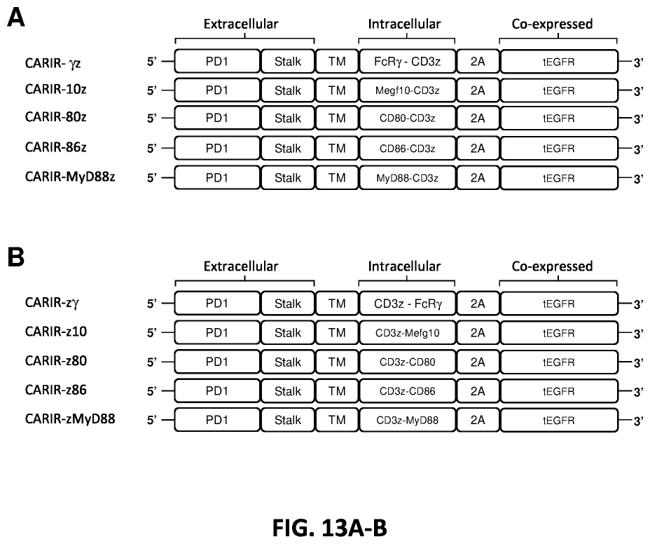

In some embodiments, the costimulatory domain is selected from the group consisting of CD80, CD86, CD40, 41BB, OX40, and CD28. Some example sequences are provided is Table 2 or illustrated in FIG. 13A-B, such as FcRγ, Megf10, CD80, CD86, and MyD88, and the intracellular domains thereof.

In some embodiments, the cytoplasmic portion of the chimeric receptor also includes a signaling/activation domain. In one embodiment, the signaling/activation domain is the CD3 domain (SEQ ID NO:39), or is an amino acid sequence having at least about 80%, 85%, 90%, 95%, 98% or 99% sequence identity to the CD3 domain.

In some embodiments, the chimeric receptor also includes a leader peptide (also referred to herein as a “signal peptide” or “signal sequence”). The inclusion of a signal sequence in a chimeric receptor is optional. If a leader sequence is included, it can be expressed on the N terminus of the chimeric receptor. Such a leader sequence can be synthesized, or it can be derived from a naturally occurring molecule. An example leader peptide is the human CSF-2 signal peptide (SEQ ID NO:26).

In some embodiments, the costimulatory domain is disposed between the transmembrane domain and the CD3 intracellular domain (FIG. 13A). In some embodiments, the costimulatory domain is disposed C-terminal to both the transmembrane domain and the CD3 intracellular domain (FIG. 13B). In some embodiment, the chimeric receptor does not include any costimulatory domain, such as in CARIR-z (FIG. 2A).

In some embodiments, the chimeric receptor of the present disclosure includes a leader peptide (P), an extracellular targeting domain (E), a hinge domain (H), a transmembrane domain (T), optionally one or more costimulatory regions (C), and an activation domain (A), wherein the chimeric receptor is configured according to any of the following: P-E-H-T-A, P-E-H-T-C-A, or P-E-H-T-A-C. In some embodiments the components of the chimeric receptor are optionally joined though a linker sequence, such as AAA or GSG. Some example sequences are provided in Table 2.

TABLE 2
Representative Elements of the Chimeric Receptor

| Elements | Sequences |
|---|---|
| human EF1α | GGCTCCGGTGCCCGTCAGTGGGCAGAGCGCACATCGCCCACAGTCCCCGAGAAGTTGG |
| promoter (SEQ | GGGGAGGGGTCGGCAATTGAACCGGTGCCTAGAGAAGGTGGCGCGGGGTAAACTGGGA |
| ID NO: 25) | AAGTGATGTCGTGTACTGGCTCCGCCTTTTTCCCGAGGGTGGGGGAGAACCGTATATA |
| | AGTGCAGTAGTCGCCGTGAACGTTCTTTTTCGCAACGGGTTTGCCGCCAGAACACAGG |
| | TAAGTGCCGTGTGTGGTTCCCGCGGGCCTGGCCTCTTTACGGGTTATGGCCCTTGCGT |
| | GCCTTGAATTACTTCCACCTGGCTGCAGTACGTGATTCTTGATCCCGAGCTTCGGGTT |
| | GGAAGTGGGTGGGAGAGTTCGAGGCCTTGCGCTTAAGGAGCCCCTTCGCCTCGTGCTT |
| | GAGTTGAGGCCTGGCCTGGGCGCTGGGGCCGCCGCGTGCGAATCTGGTGGCACCTTCG |
| | CGCCTGTCTCGCTGCTTTCGATAAGTCTCTAGCCATTTAAAATTTTTGATGACCTGCT |
| | GCGACGCTTTTTTTCTGGCAAGATAGTCTTGTAAATGCGGGCCAAGATCTGCACACTG |
| | GTATTTCGGTTTTTGGGGCCGCGGGCGGCGACGGGGCCCGTGCGTCCCAGCGCACATG |
| | TTCGGCGAGGCGGGGCCTGCGAGCGCGGCCACCGAGAATCGGACGGGGGTAGTCTCAA |
| | GCTGGCCGGCCTGCTCTGGTGCCTGGTCTCGCGCCGCCGTGTATCGCCCCGCCCTGGG |
| | CGGCAAGGCTGGCCCGGTCGGCACCAGTTGCGTGAGCGGAAAGATGGCCGCTTCCCGG |
| | CCCTGCTGCAGGGAGCTCAAAATGGAGGACGCGGCGCTCGGGAGAGCGGGCGGGTGAG |
| | TCACCCACACAAAGGAAAAGGGCCTTTCCGTCCTCAGCCGTCGCTTCATGTGACTCCA |
| | CGGAGTACCGGGCGCCGTCCAGGCACCTCGATTAGTTCTCGAGCTTTTGGAGTACGTC |
| | GTCTTTAGGTTGGGGGGAGGGGTTTTATGCGATGGAGTTTCCCCACACTGAGTGGGTG |
| | GAGACTGAAGTTAGGCCAGCTTGGCACTTGATGTAATTCTCCTTGGAATTTGCCCTTT |
| | TTGAGTTTGGATCTTGGTTCATTCTCAAGCCTCAGACAGTGGTTCAAAGTTTTTTTCT |
| | TCCATTTCAGGTGTCGTGA |
| human CSF-2 | MWLQSLLLLGTVACSIS |
| signal peptide | |
| (SEQ ID NO: 26) | |
| human CD8α | KPTTTPAPRPPTPAPTIASQPLSLRPEACRPAAGGAVHTRGLDFACDIY |
| hinge (SEQ ID | |
| NO: 27) | |
| human CD8α | IWAPLAGTCGVLLLSLVITLY |
| transmembrane | |
| domain (SEQ ID | |
| NO: 28) | |
| P2A protease | ATNFSLLKQAGDVEENPGP |
| site (SEQ ID | |
| NO: 29) | |
| T2A protease | EGRGSLLTCGDVEENPGP |
| site (SEQ ID | |
| NO: 30) | |
| human CD40 | KKVAKKPTNKAPHPKQEPQEINFPDDLPGSNTAAPVQETLHGCQPVTQEDGKESRISV |
| isoform 1 | QERQ |
| cytoplasmic | |
| domain (SEQ ID | |
| NO: 31) | |
| human IL-12 | Coding sequence (SEQ ID NO: 32) |
| fusion (codon | ATGTGCCACCAGCAACTTGTAATAAGTTGGTTCTCACTGGTCTTCCTTGCGAGCCCGT |
| optimized) | TGGTAGCAATCTGGGAACTGAAAAAGGACGTATATGTGGTGGAGCTCGATTGGTATCC |
| | CGACGCACCCGGGGAGATGGTTGTCCTCACCTGTGATACCCCAGAGGAAGACGGTATC |
| | ACTTGGACCTTGGATCAGTCCTCCGAGGTGCTTGGAAGCGGAAAAACTCTGACGATAC |
| | AGGTGAAAGAATTCGGCGACGCGGGACAGTATACATGCCACAAAGGCGGGGAGGTACT |
| | GTCACATTCACTCCTGCTCTTGCATAAAAAGGAGGACGGGATCTGGAGCACTGATATT |
| | TTGAAAGACCAAAAAGAACCAAAAAACAAGACCTTCCTCAGGTGTGAGGCCAAGAATT |
| | ATAGTGGACGCTTCACCTGCTGGTGGCTGACCACAATTAGTACTGACTTGACTTTCTC |
| | AGTAAAGAGTTCCAGGGGGAGTAGTGACCCCCAAGGGGTAACCTGCGGGGCCGCGACT |
| | TTGTCAGCGGAACGGGTGCGAGGGGATAATAAGGAATATGAGTATTCAGTGGAGTGCC |
| | AGGAAGACTCTGCATGTCCCGCAGCAGAGGAAAGCCTGCCGATAGAGGTAATGGTTGA |
| | CGCCGTCCATAAGCTCAAATACGAGAACTACACAAGCTCTTTCTTCATACGGGACATT |
| | ATTAAGCCAGACCCCCCGAAGAATCTTCAACTTAAACCGTTGAAAAATAGTCGACAAG |
| | TGGAAGTCAGTTGGGAATATCCAGACACCTGGAGTACGCCACACAGCTATTTTTCCTT |
| | GACATTTTGCGTCCAGGTTCAGGGGAAATCAAAGCGCGAGAAGAAGGATCGAGTCTTT |
| | ACAGACAAGACGAGTGCAACGGTAATCTGTAGGAAGAACGCAAGCATTTCCGTCAGAG |
| | CTCAGGACCGGTACTATAGCAGTTCATGGTCTGAATGGGCTAGTGTACCTTGCAGTGG |
| | AGGTGGCGGCGGCGGCTCTCGAAATCTGCCAGTCGCTACCCCGGACCCAGGAATGTTT |
| | CCATGCCTGCACCACAGTCAGAACCTGCTCCGGGCGGTTTCCAACATGCTTCAGAAAG |
| | CGCGCCAGACCCTTGAATTTTACCCCTGCACAAGTGAAGAGATAGACCATGAAGATAT |
| | TACCAAGGATAAAACATCAACTGTAGAGGCGTGTCTCCCTCTCGAACTGACAAAGAAC |
| | GAGTCTTGTCTCAATAGTAGGGAAACTTCATTCATTACAAACGGGTCATGTCTTGCTT |
| | CAAGGAAGACCAGCTTCATGATGGCACTCTGCTTGTCTTCAATCTATGAGGATCTTAA |
| | AATGTACCAAGTAGAGTTTAAGACTATGAATGCGAAGCTCCTGATGGATCCGAAGCGG |
| | CAGATTTTTTTGGACCAGAATATGTTGGCGGTCATTGACGAACTTATGCAAGCTCTCA |
| | ATTTCAATTCAGAGACGGTTCCTCAGAAAAGCTCCTTGGAAGAGCCGGACTTCTACAA |
| | AACTAAGATCAAATTGTGTATCTTGCTCCATGCATTCCGGATACGCGCCGTGACCATT |
| | GATCGAGTAATGTCCTATTTGAATGCAAGCTAA |
| | Amino acid sequence (SEQ ID NO: 33) |
| | MCHQQLVISWFSLVFLASPLVAIWELKKDVYVVELDWYPDAPGEMVVLTCDTPEEDGI |
| | TWTLDQSSEVLGSGKTLTIQVKEFGDAGQYTCHKGGEVLSHSLLLLHKKEDGIWSTDI |
| | LKDQKEPKNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYTSSFFIRDI |
| | IKPDPPKNLQLKPLKNSRQVEVSWEYPDTWSTPHSYFSLTFCVQVQGKSKREKKDRVF |
| | TDKTSATVICRKNASISVRAQDRYYSSSWSEWASVPCSGGGGGGSRNLPVATPDPGMF |
| | PCLHHSQNLLRAVSNMLQKARQTLEFYPCTSEEIDHEDITKDKTSTVEACLPLELTKN |
| | ESCLNSRETSFITNGSCLASRKTSFMMALCLSSIYEDLKMYQVEFKTMNAKLLMDPKR |
| | QIFLDQNMLAVIDELMQALNENSETVPQKSSLEEPDFYKTKIKLCILLHAFRIRAVTI |
| | DRVMSYLNAS |
| human IL-12B/ | Coding sequence (SEQ ID NO: 34) |
| p40 (codon | ATGTGCCACCAGCAACTTGTAATAAGTTGGTTCTCACTGGTCTTCCTTGCGAGCCCGT |
| optimized) | TGGTAGCAATCTGGGAACTGAAAAAGGACGTATATGTGGTGGAGCTCGATTGGTATCC |
| | CGACGCACCCGGGGAGATGGTTGTCCTCACCTGTGATACCCCAGAGGAAGACGGTATC |
| | ACTTGGACCTTGGATCAGTCCTCCGAGGTGCTTGGAAGCGGAAAAACTCTGACGATAC |
| | AGGTGAAAGAATTCGGCGACGCGGGACAGTATACATGCCACAAAGGCGGGGAGGTACT |
| | GTCACATTCACTCCTGCTCTTGCATAAAAAGGAGGACGGGATCTGGAGCACTGATATT |
| | TTGAAAGACCAAAAAGAACCAAAAAACAAGACCTTCCTCAGGTGTGAGGCCAAGAATT |
| | ATAGTGGACGCTTCACCTGCTGGTGGCTGACCACAATTAGTACTGACTTGACTTTCTC |
| | AGTAAAGAGTTCCAGGGGGAGTAGTGACCCCCAAGGGGTAACCTGCGGGGCCGCGACT |
| | TTGTCAGCGGAACGGGTGCGAGGGGATAATAAGGAATATGAGTATTCAGTGGAGTGCC |
| | AGGAAGACTCTGCATGTCCCGCAGCAGAGGAAAGCCTGCCGATAGAGGTAATGGTTGA |
| | CGCCGTCCATAAGCTCAAATACGAGAACTACACAAGCTCTTTCTTCATACGGGACATT |
| | ATTAAGCCAGACCCCCCGAAGAATCTTCAACTTAAACCGTTGAAAAATAGTCGACAAG |
| | TGGAAGTCAGTTGGGAATATCCAGACACCTGGAGTACGCCACACAGCTATTTTTCCTT |
| | GACATTTTGCGTCCAGGTTCAGGGGAAATCAAAGCGCGAGAAGAAGGATCGAGTCTTT |
| | ACAGACAAGACGAGTGCAACGGTAATCTGTAGGAAGAACGCAAGCATTTCCGTCAGAG |
| | CTCAGGACCGGTACTATAGCAGTTCATGGTCTGAATGGGCTAGTGTACCTTGCAGT |
| | Amino acid sequence (SEQ ID NO: 35) |
| | MCHQQLVISWFSLVFLASPLVAIWELKKDVYVVELDWYPDAPGEMVVLTCDTPEEDGI |
| | TWTLDQSSEVLGSGKTLTIQVKEFGDAGQYTCHKGGEVLSHSLLLLHKKEDGIWSTDI |
| | LKDQKEPKNKTFLRCEAKNYSGRFTCWWLTTISTDLTFSVKSSRGSSDPQGVTCGAAT |
| | LSAERVRGDNKEYEYSVECQEDSACPAAEESLPIEVMVDAVHKLKYENYTSSFFIRDI |
| | IKPDPPKNLQLKPLKNSRQVEVSWEYPDTWSTPHSYFSLTFCVQVQGKSKREKKDRVF |
| | TDKTSATVICRKNASISVRAQDRYYSSSWSEWASVPCS |
| G₆S linker (SEQ | GGGGGGS |
| ID NO: 36) | |
| human IL-12A/ | Coding sequence (SEQ ID NO: 37) |
| p35 (codon | CGAAATCTGCCAGTCGCTACCCCGGACCCAGGAATGTTTCCATGCCTGCACCACAGTC |
| optimized) | AGAACCTGCTCCGGGCGGTTTCCAACATGCTTCAGAAAGCGCGCCAGACCCTTGAATT |
| | TTACCCCTGCACAAGTGAAGAGATAGACCATGAAGATATTACCAAGGATAAAACATCA |
| | ACTGTAGAGGCGTGTCTCCCTCTCGAACTGACAAAGAACGAGTCTTGTCTCAATAGTA |
| | GGGAAACTTCATTCATTACAAACGGGTCATGTCTTGCTTCAAGGAAGACCAGCTTCAT |
| | GATGGCACTCTGCTTGTCTTCAATCTATGAGGATCTTAAAATGTACCAAGTAGAGTTT |
| | AAGACTATGAATGCGAAGCTCCTGATGGATCCGAAGCGGCAGATTTTTTTGGACCAGA |
| | ATATGTTGGCGGTCATTGACGAACTTATGCAAGCTCTCAATTTCAATTCAGAGACGGT |
| | TCCTCAGAAAAGCTCCTTGGAAGAGCCGGACTTCTACAAAACTAAGATCAAATTGTGT |
| | ATCTTGCTCCATGCATTCCGGATACGCGCCGTGACCATTGATCGAGTAATGTCCTATT |
| | TGAATGCAAGCTAA |
| | Amino acid sequence (SEQ ID NO: 38) |
| | RNLPVATPDPGMFPCLHHSQNLLRAVSNMLQKARQTLEFYPCTSEEIDHEDITKDKTS |
| | TVEACLPLELTKNESCLNSRETSFITNGSCLASRKTSFMMALCLSSIYEDLKMYQVEF |
| | KTMNAKLLMDPKRQIFLDQNMLAVIDELMQALNENSETVPQKSSLEEPDFYKTKIKLC |
| | ILLHAFRIRAVTIDRVMSYLNAS |
| CD3ξ domain | RVKFSRSADAPAYQQGQNQLYNELNLGRREEYDVLDKRRGRDPEMGGKPRRKNPQEGL |
| (SEQ ID NO: 39) | YNELQKDKMAEAYSEIGMKGERRRGKGHDGLYQGLSTATKDTYDALHMQALPPR |
| Truncated EGFR | RKVCNGIGIGEFKDSLSINATNIKHFKNCTSISGDLHILPVAFRGDSFTHTPPLDPQE |
| (SEQ ID NO: 40) | LDILKTVKEITGFLLIQAWPENRTDLHAFENLEIIRGRTKQHGQFSLAVVSLNITSLG |
| | LRSLKEISDGDVIISGNKNLCYANTINWKKLFGTSGQKTKIISNRGENSCKATGQVCH |
| | ALCSPEGCWGPEPRDCVSCRNVSRGRECVDKCNLLEGEPREFVENSECIQCHPECLPQ |
| | AMNITCTGRGPDNCIQCAHYIDGPHCVKTCPAGVMGENNTLVWKYADAGHVCHLCHPN |
| | CTYGCTGPGLEGCPTNGPKIPSIATGMVGALLLLLVVALGIGLFM |

### Engineered Immune Cells

The chimeric receptors disclosed herein can be expressed in an immune cell which can be suitably used for therapeutic purposes. Example immune cells include myeloid cells, natural killer (NK) cells, T cells, tumor infiltrating lymphocytes, and natural killer T (NKT) cells. The preparation and use of T cells transduced to express chimeric antigen receptors (CAR) have been well described in the ant. The instantly disclosed chimeric receptors can likewise be expressed in T cells, and are used like CAR-T cells. Nevertheless, the present technology is not limited to T cells.

In some embodiments, the immune cell is a myeloid cell, in particular an immature myeloid cell (IMC).

Myeloid cells are produced by hematopoietic stem cells. Myeloid cells are progenitor cells which can produce different types of blood cells including monocytes, macrophages, neutrophils, basophils, eosinophils, erythrocytes, dendritic cells, megakaryocytes, and platelets. Myeloid cells originate in bone marrows. Myeloid cells encompass circulating progenitor monocytes and tissue resident macrophage cells, including hepatic Kupffer cells, lymph-associated macrophages in spleen and lymph nodes, Langerhans cells in the skin, pulmonary alveolar macrophages, and highly specialized dendritic cells found primarily along mucosal surfaces.

“Immature myeloid cells” (IMC), “early myeloid cells,” “myeloid suppressive cells,” or “myeloid-derived suppressor cells” (MDSCs), are progenitor cells present in the bone marrow and spleen of healthy subjects which can differentiate into mature myeloid cells under normal conditions. These cells are associated with immune suppression during viral infection, transplantation, UV irradiation and cyclophosphamide (CTX) treatment. It has also been shown that the accumulation of IMC within the tumor microenvironment correlates with a poor prognosis. The instant inventors have the insight that immunotherapy with such cells can promote penetration into the tumor microenvironment.

The IMC can be prepared using established methods from selected autologous cell sources, such as CD34+ hematopoietic stem cells from the bone marrow or mobilized CD34+ hematopoietic stem cells from the peripheral blood. Alternatively, the IMC can be generated in vitro from induced pluripotent stem cells (iPSCs).

In some embodiments, the immune cell is engineered to be p50 deficient. NF-κB p50 (nuclear factor NF-kappa-B p105 subunit) is a Rel protein-specific transcription inhibitor, and is the DNA binding subunit of the NF-kappaB (NF-κB) protein complex. NF-κB is a transcription factor that is activated by various intra- and extra-cellular stimuli such as cytokines, oxidant-free radicals, ultraviolet irradiation, and bacterial or viral products. Activated NF-κB translocates into the nucleus and stimulates the expression of genes involved in a wide variety of biological functions. p50 is an inhibitory subunit; in the basal state p65 is held in the cytoplasm by IκB, whereas p50:p50 homo-dimers enter the nucleus, bind DNA, and repress gene expression. Absence of p50 leads to activation of pro-inflammatory pathways.

In some embodiments, a p50 deficient immune cell is an immune cell that has been engineered to have reduced expression or biological activity of the p50 gene. In some embodiments, a p50 deficient immune cell is an immune cell in which the p50 gene is knocked out (p50−/−). Reduced expression or biological activity or knock-out can be readily implemented with techniques well known in the art, such as CRISPR. In some embodiments, a single allele of the p50 gene is inactivated; in some embodiments, both alleles of the p50 gene are inactivated. In some embodiments, the immune cell is a p50^{-/-} immature myeloid cell.

In some embodiments, the immune cell is further engineered to produce a proinflammatory cytokine. Example proinflammatory cytokines include the IL-1 family (e.g., IL18, IL18BP, IL1A, IL1B, IL1F10, IL1F3/IL1RA, IL1F5, IL1F6, IL1F7, IL1F8, IL1RL2, IL1F9, and IL33), IL-1 receptors (e.g., IL18R1, IL18RAP, IL1R1, IL1R2, IL1R3, IL1R8, IL1R9, IL1RL1, and SIGIRR), IL-15, the TNF family (BAFF, 4-1BBL, TNFSF8, CD40LG, CD70, CD95L/CD178, EDA-A1, TNFSF14, LTA/TNFB, LTB, TNFa, TNFSF10, TNFSF11, TNFSF12, TNFSF13, TNFSF15, and TNFSF4), TNF receptors (e.g., 4-1BB, BAFFR, TNFRSF7, CD40, CD95, DcR3, TNFRSF21, EDA2R, EDAR, PGLYRP1, TNFRSF19L, TNFR1, TNFR2, TNFRSF11A, TNFRSF11B, TNFRSF12A, TNFRSF13B, TNFRSF14, TNFRSF17, TNFRSF18, TNFRSF19, TNFRSF25, LTBR, TNFRSF4, TNFRSF8, TRAILR1, TRAILR2, TRAILR3, and TRAILR4), Interferons (IFN) (e.g., IFNA1, IFNA10, IFNA13, IFNA14, IFNA2, IFNA4, IFNA7, IFNB1, IFNE, IFNG, IFNZ, IFNA8, IFNA5/IFNaG, and IFNω/IFNW1), IFN receptors (e.g., IFNAR1, IFNAR2, IFNGR1, and IFNGR2), the IL6 family (e.g., CLCF1, CNTF, IL11, IL31, IL6, Leptin, LIF, OSM, IL6 Receptor, CNTFR, IL11RA, IL6R, LEPR, LIFR, OSMR, and IL31RA), the IL10 family (e.g., IL10, IL19, IL20, IL22, IL24, IL28B, IL28A, and IL29), the IL10 family receptors (e.g., IL10RA, IL10RB, IL20RA, IL20RB, IL22RA2, and IL22R), the TGF beta family (e.g., TGF-beta 1/TGFB1, TGF-beta 2/TGFB2, and TGF-beta 3/TGFB3), TGF beta family receptors (e.g., ALK-7, ATF2, CD105/ENG, TGFBR1, TGFBR2, and TGFBR3), chemokines (e.g., CCL1/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, XCL1, XCL2, FAM19A1, FAM19A2, FAM19A3, FAM19A4, and FAM19A5), and chemokine receptors (e.g., CCR1, CCR2, CCR3, CCR4, CCR5, CCR6, CCR7, CCR8, CCRL1, CXCR3, CXCR4, CXCR5, CXCR6, CXCR7, CXCR1, and CXCR2).

In some embodiments, the proinflammatory cytokine is IL-12, IFN-γ, TNF-α, and/or IL-1β. In some embodiments, the expression of the cytokine is constitutive. In some embodiments, the expression of the cytokine is inducible.

It is contemplated that expression of the proinflammatory cytokine, such as IL-12, enable the engineered immune cells to reprogram and overcome the immunosuppressive TME. Further, the additional expression of the proinflammatory cytokine can enhance tumor antigen presentation, increase tumor-specific cytotoxic T cells activation, and prevent or reduce tumor metastasis.

In some embodiments, the immune cell further expresses a kill switch (or safety module). The kill switch allows the engineered immune cell to be killed or turned off when needed. In some embodiments, the kill switch is a human HSV-TK, a truncated EGFR (tEGFR, e.g., SEQ ID NO:40), CD19, or a CD20 protein or fragment. In the case of unacceptable toxicity, the immune cells can be eliminated through administration of a corresponding drug (e.g., ganciclovir) or depleting antibody, (e.g., cetuximab or rituximab).

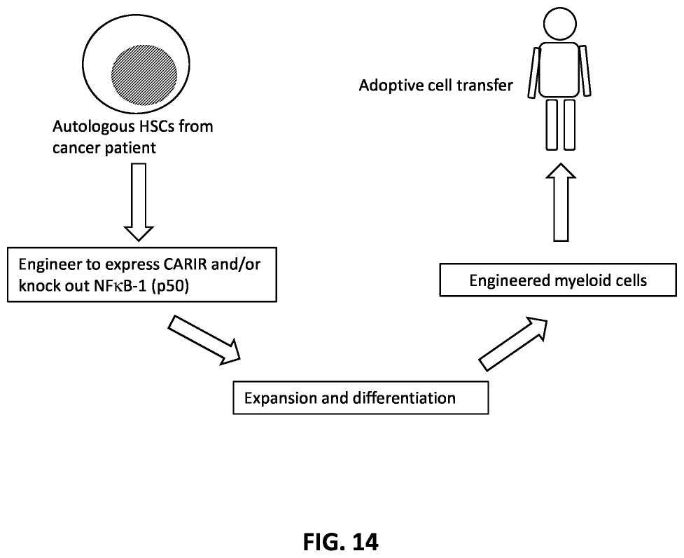

An example adoptive engineered-myeloid cell therapy for treating cancer is illustrated in FIG. 14. At a first step, autologous HSCs are acquired from a cancer patient. Within the HSCs, the p50 gene is inactivated and a CARIR construct is introduced. Then, the transduced HSCs are optionally expanded and differentiated into immature myeloid cells (IMC) or other types of myeloid cells or immune cells, which are transferred back to the patient for treatment.

### Polynucleotides and Vectors

The present disclosure also provides polynucleotides or nucleic acid molecules encoding the chimeric receptor, optionally along with other useful components of the engineered immune cell (e.g., proinflammatory cytokine and/or kill switch).

The polynucleotides of the present disclosure may encode chimeric receptor, the proinflammatory cytokine and kill switch on the same polynucleotide molecule (as exemplified in FIG. 1) or on separate polynucleotide molecules.

As illustrated in FIG. 1, the vector encodes a preprotein that includes a leader peptide from EF1α, a chimeric receptor that includes an extracellular domain of PD1, a CD8 hinge region, a CD8 transmembrane domain, a CD40 costimulatory domain, the CD activation domain, a protease digestion site P2A, a kill switch (truncated EGFR (tEGFR)), a protease digestion site T2A, and an IL-12 (including p40 and p35, connected through a short G₆S linker (SEQ ID NO:36) linker) as the additional proinflammatory cytokine. Upon protease treatments, the preprotein can be split into the chimeric receptor, the tEGFR kill switch, and the IL-12 cytokine.

The polynucleotides encoding desired proteins may be readily prepared, isolated and sequenced using conventional procedures (e.g., by using oligonucleotide probes that are capable of binding specifically to genes encoding the receptor).

Additionally, standard techniques known to those of skill in the art can be used to introduce mutations in the nucleotide sequence encoding a chimeric receptor of the present disclosure, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference chimeric receptor.

The polynucleotides and vectors of the present disclosure can be introduced to a target immune cell with techniques known in the art.

### Cancer Treatment

As described herein, the engineered immune cells of the present disclosure can be used in certain treatment methods. Accordingly, one embodiment of the present disclosure is directed to immune cell-based therapies which involve administering the immune cells of the disclosure to a patient such as an animal, a mammal, and a human for treating one or more of the disorders or conditions described herein.

In some embodiments, the cancer being treated expresses a ligand corresponding to the extracellular targeting domain of the chimeric receptor. For instance, if the cancer cells express PD-L1, then a suitable chimeric receptor includes an extracellular targeting domain of PD1. Likewise, if the cancer cells express CD47, then a suitable chimeric receptor includes an extracellular targeting domain of SIRPα.

Cancers that can be suitable treated by the present technology include bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer. Accordingly, the presently disclosed antibodies can be used for treating any one or more such cancers, in particular non-small cell lung cancer (NSCLC), small cell lung cancer, prostate cancer, pancreatic ductal carcinoma, neuroblastoma, glioblastoma, ovarian cancer, melanoma, and breast cancer. In some embodiments, the cancer is metastatic cancer.

Additional diseases or conditions associated with increased cell survival, that may be treated or prevented include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, the immune cell is isolated from the cancer patient him- or her-self. In some embodiments, the immune cell is provided by a donor or from a cell bank. When the cell is isolated from the cancer patient, undesired immune reactions can be minimized. The isolated immune cell can then be transduced with the polynucleotides or vectors of the present disclosure to prepare the engineered immune cell.

### Combination Therapies

In a further embodiment, the compositions of the disclosure are administered in combination with a different antineoplastic agent. Any of these agents known in the art may be administered in the compositions of the current disclosure.

In one embodiment, compositions of the disclosure are administered in combination with a chemotherapeutic agent. Chemotherapeutic agents that may be administered with the compositions of the disclosure include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

Combination therapies are also provided, which includes the use of one or more of the immune cells of the present disclosure along with a second anticancer (chemotherapeutic) agent. Chemotherapeutic agents may be categorized by their mechanism of action into, for example, anti-metabolites/anti-cancer; purine analogs, folate antagonists, and related inhibitors, antiproliferative/antimitotic agents, DNA damaging agents, antibiotics, enzymes such as L-asparaginase which systemically metabolizes L-asparagine and deprives cells which do not have the capacity to synthesize their own asparagine, antiplatelet agents, antiproliferative/antimitotic alkylating agents, antiproliferative/antimitotic antimetabolites, platinum coordination complexes, hormones, hormone analogs, anticoagulants, fibrinolytic agents, antimigratory agents, antisecretory agents, immunosuppressives, angiotensin receptor blockers, nitric oxide donors, cell cycle inhibitors and differentiation inducers, topoisomerase inhibitors, growth factor signal transduction kinase inhibitors, without limitation.

Additional examples include alkylating agents, alkyl sulfonates, aziridines, emylerumines and memylamelamines, acetogenins, nitrogen mustards, nitrosoureas, anti-metabolites, folic acid analogs, purine analogs, pyrimidine analogs, androgens, anti-adrenals, folic acid replinishers, trichothecenes, and taxoids, platinum analogs.

In one embodiment, the compounds and compositions described herein may be used or combined with one or more additional therapeutic agents. The one or more therapeutic agents include, but are not limited to, an inhibitor of Abl, activated CDC kinase (ACK), adenosine A2B receptor (A2B), apoptosis signal-regulating kinase (ASK), Auroa kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, FYN, glycogen synthase kinase (GSK), HCK, histone deacetylase (HDAC), IKK such as IKKβε, isocitrate dehydrogenase (IDH) such as IDH1, Janus kinase (JAK), KDR, lymphocyte-specific protein tyrosine kinase (LCK), lysyl oxidase protein, lysyl oxidase-like protein (LOXL), LYN, matrix metalloprotease (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase STK, signal transduction and transcription (STAT), SRC, serine/threonine-protein kinase (TBK) such as TBK1, TIE, tyrosine kinase (TK), vascular endothelial growth factor receptor (VEGFR), YES, or any combination thereof.

In some embodiments, the compositions of the disclosure are used in combination with a checkpoint inhibitor, such as anti-CTLA-4 or anti-PD-1/PD-L1 (for non PD-1 CARIR) blockade antibody. In some embodiments, the compositions of the disclosure are used in combination with CAR-T or CAR-NK cells etc.

For any of the above combination treatments, the engineered immune cell can be administered concurrently or separately from the other anticancer agent. When administered separately, the engineered immune cell can be administered before or after the other anticancer agent.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Preparation and Expression of CAR-Like Immune Receptors (CARIR)

This example demonstrates the design, construction, and expression of a CARIR that contained a PD-1 extracellular domain.

The structure of the preprotein for the CARIR molecule is illustrated in FIG. 1. The preprotein includes a leader peptide from EF1α, a chimeric receptor that includes an extracellular domain of PD-1 (SEQ ID NO:3), a CD8 hinge region (SEQ ID NO:27), a CD8 transmembrane domain (SEQ ID NO:28), a CD40 costimulatory domain (SEQ ID NO:31), the CD3 activation domain (SEQ ID NO:39), a protease digestion site P2A (SEQ ID NO:29), a kill switch (truncated EGFR (tEGFR); SEQ ID NO:40), a protease digestion site T2A (SEQ ID NO:30), and an IL-12 (SEQ ID NO:33, including p40 (SEQ ID NO:35) and p35 (SEQ ID NO:38), connected through a short G₆S linker (SEQ ID NO:36) linker) as the additional proinflammatory cytokine. Upon protease treatments, the preprotein can be split into the chimeric receptor, the tEGFR kill switch, and the IL-12 cytokine.

Variants of this preprotein structure were also prepared, as illustrated in FIG. 2A. As compared to CARIR-40z-12, variant CARIR-Δz does not include the CD40 costimulatory domain, the CD3 activation domain, and the cytokine IL-12; variant CARIR-z does not include the CD40 costimulatory domain and IL-12; variant CARIR-z-12 does not include the CD40 costimulatory domain; variant CARIR-40 does not include the CD3 activation domain and the cytokine IL-12; and variant CARIR-40z does not include the cytokine IL-12.

Synthesized cDNA expressing each of these variant was inserted into a lentiviral vector, which was transduced to human monocytic THP-1 cells. As shown in FIG. 2B, lentiviral mediated CARIR transduction in human monocytic THP-1 cells with decreasing multiplicity of infection (MOI). FIG. 2C shows lentiviral mediated CARIR expression in human monocytic THP-1 cells at MOI of 10. All of these constructs showed excellent expression in the THP-1 cells.

### Example 2. CARIR Bound to PD-L1 and Upregulated CD86 and CD80

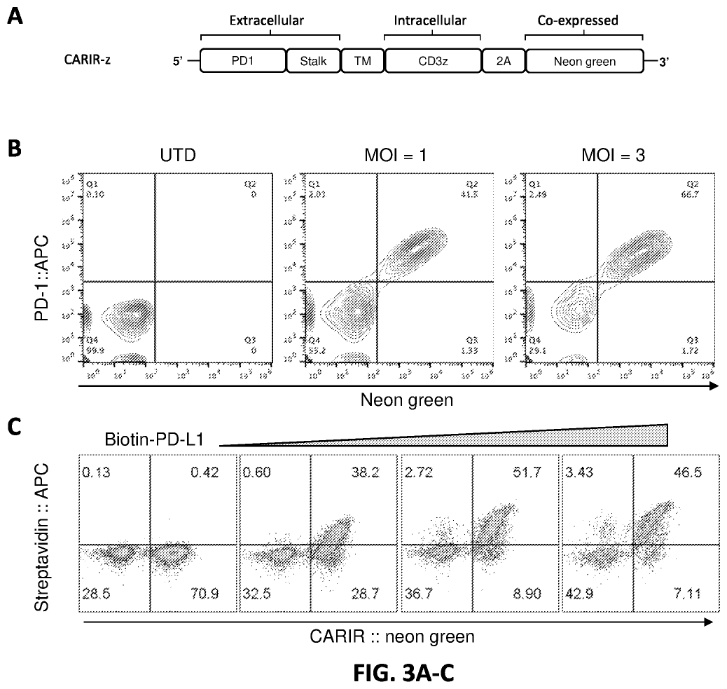

One of the variants, CARIR-z, was subjected to binding tests. In addition to CARIR-z, the construct further included a Neon green marker (FIG. 3A). Its expression in the transduced human monocytic THP-1 cells was measured by flow cytometry (FIG. 3B). The transduced THP-1 cells were then incubated with biotinylated human PD-L1 protein. As shown in FIG. 3C, the THP-1 cells bound to the biotinylated human PD-L1.

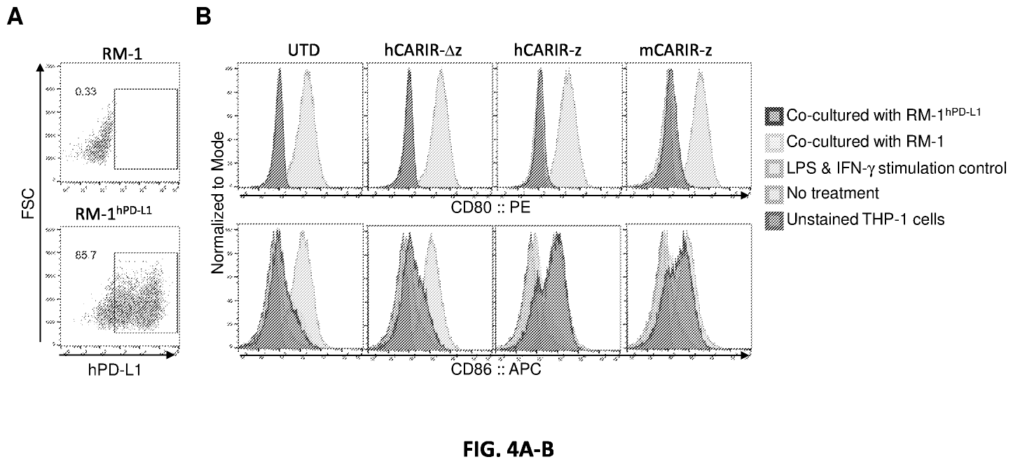

It was then tested whether such binding triggers the proper biological signaling. The CARIR-Δz and CARIR-z constructs were used in this assay, along with a mouse counterpart, mCARIR-z which contained the extracellular domain of the mouse PD-1 protein. RM-1 cells transduced with the human PD-L1 protein (RM-1^{hPD-L1}) were confirmed to express PD-L1 (FIG. 4A). The THP-1 cells transduced with the CARIR constructs were then stimulated with 1 ng/ml PMA for 24 hours, followed by the indicated co-culture treatment for 3 days. When the CARIR-expressing THP-1 cells were incubated with the RM-1^{hPD-L1} cells, upregulation of CD86 and, to a much lesser degree, CD80, was observed (FIG. 4B).

### Example 3. CARIR Enhanced Target Cell Phagocytosis

This example tested whether the CARIR protein expressed in monocytic THP-1 cells can enhance target cell phagocytosis.

The effector cells were prepared from THP-1 cells that were not transduced (THP-1), or lentivirally transduced to express CARIR-Δz (CARIR-Δz THP-1) or and CARIR-z (CARIR-z THP-1). The target cells were either wild type RM-1 cells (RM-1) or the RM-1 cells that were lentivirally transduced to overexpress human PD-L1 (RM-1^{hPD-L1}). The ratio of effector and target cells was at 5:1 during the co-culture (FIG. 5A).

Flow analysis was used to detect phagocytosis events. The cells were gated on FSC/SSC, singlets, and viable cells. Dot plots showing phagocytosis events in the Q2 quadrant, which is CellTrace CFSE and violet double positive (FIG. 5B). The bar graphs in FIG. 5C summarizes that CARIR expression significantly enhanced the phagocytosis of the target cells. The data are expressed as mean±SEM. ****p<0.0001, one-way ANOVA compared to the CARIR-z THP-1+RM-1^{hPD-L1} group. Among them, CARIR-z exhibited the highest efficacy.

### Example 4. CARIR-Expressing Immature Myeloid Cells Inhibited Tumor Growth

This example tested CARIR-expressing cells' ability to inhibit tumor growth.

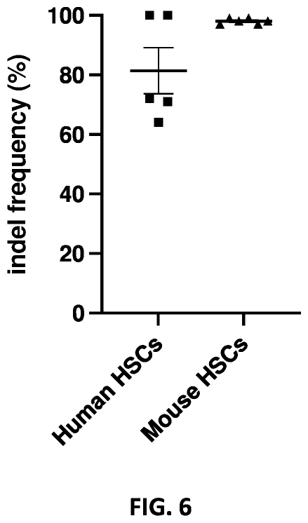

Mouse immature myeloid cells (IMC) were derived from mouse hematopoietic stem cells (HSCs) in which the NFκB-1 (p50) gene was knocked-out. The knock-out was achieved with the CRISPR/Cas9 technology (FIG. 6) which had efficient CRISPR/Cas9 mediated p50 gene knockout in human and mouse HSCs.

Mouse IMC transduced with a murine analogue of CARIR-z (CARIR-IMC) were infused to a syngeneic mouse model. The experimental timeline is shown in FIG. 7A. Syngeneic Balb/c mice were subcutaneously implanted with 5×10⁴ 4T1 breast cancer cells on day −7. Starting on day 0, the mice were treated with 3 weekly dose of IMC (10×10⁶ for each dose) or PBS as indicated.

FIG. 7B shows the results of tumor volume measurements and FIG. 7C shows probability of survival, demonstrating the efficacy of CARIR-IMC. FIG. 7D shows body weight over the course of the experiment, demonstrating safety of the treatments. These data, therefore, show that p50^{-/-}-IMC slowed 4T1 tumor growth and prolonged survival in the syngeneic mouse model.

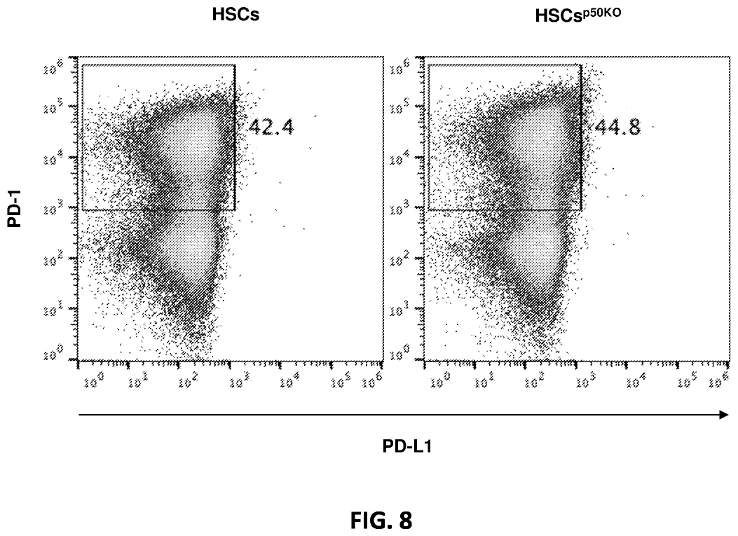

Human hematopoietic stem cells (HSCs) with a p50 knock-out were also prepared by CRISPR/Cas9 with human CD34+ HSCs isolated from mobilized peripheral blood. Non-modified human HSCs or HSCs^{p50KO} were transduced with lentiviral vector encoding CARIR-z at the MOI of 10. Five days following transduction, CARIR expression was evaluated by flow cytometry based on PD-1 expression. As shown in FIG. 8, NFκB-1 (p50) knock-out in did not affect CARIR-z expression in these human cells.

### Example 5. CARIR in Other Cells

This example tested the transduction of CARIR in various types of cells.

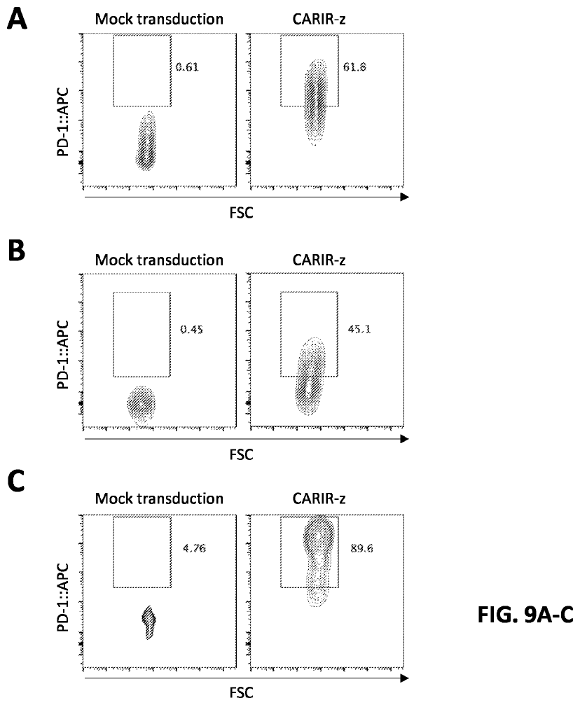

Human PBMC isolated from healthy donor were stimulated with anti-CD3/CD28 for 24 hrs in the presence of 200 U/ml human recombinant IL-2. The activated cells were then transduced with CARIR-z lentiviral vector at MOI of 25 through spinoculation. Three days following the spinoculation, the percentage of CARIR expression among T cells (CD3+), NK cells (CD3-CD56+), and NKT cells (CD3+CD56+) was analyzed based on PD-1 expression by flow cytometry. The results are presented in FIG. 9A (CD3+ T cells), 9B (CD3-CD56+NK cells) and 9C (CD3+CD56+ NKT cells).

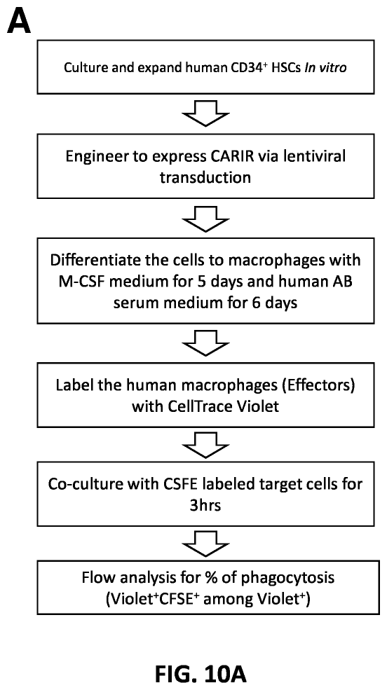

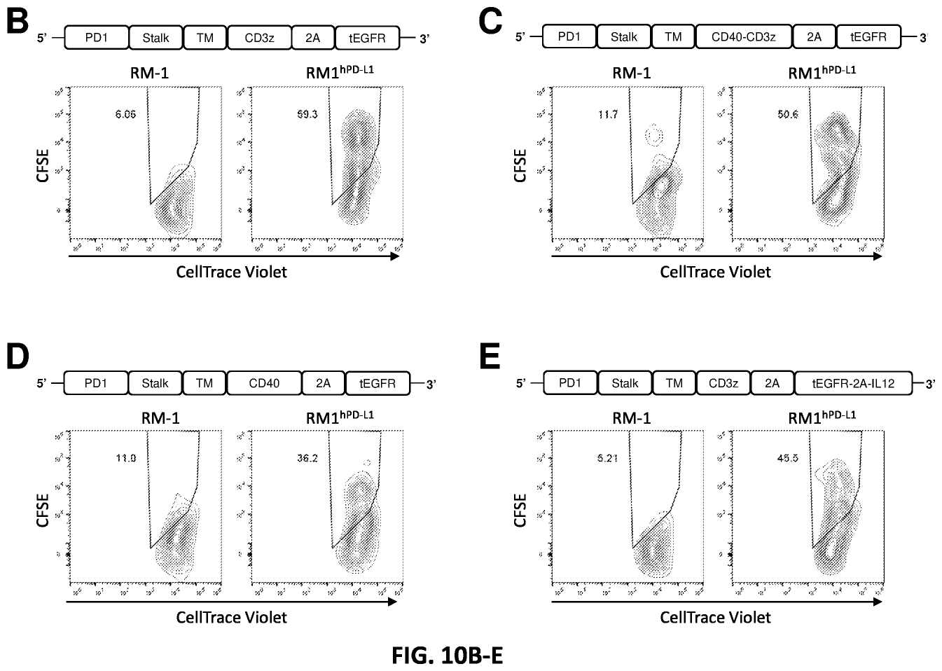

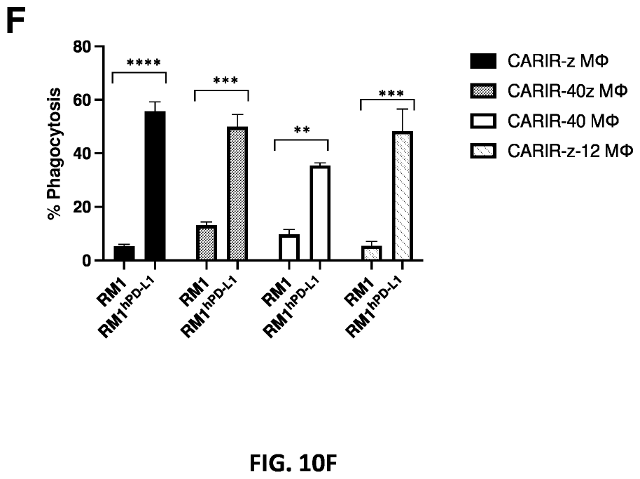

Next, it was shown that CARIR modified human macrophages can efficiently phagocytose target cells that express human PD-L1. For generating CARIR modified human macrophages to serve as the effector cells, human CD34+ hematopoietic stem cells (HSCs) were engineered to express CARIR through lentiviral transduction, and then differentiated into macrophages. For target cells, both wild type RM1 cells and RM1^{hPD-L1} cells were used. The latter was established by overexpressing human PD-L1 through lentiviral vector-mediated transduction (FIG. 10A). FIG. 10B-E show flow cytometry data showing the % phagocytosis of human CARIR-z macrophages (10B), CARIR-40z macrophages (10C), CARIR-40 macrophages (10D), and CARIR-40 macrophages (10E) on wild RM1 or RM1^{hPD-L1} target cells. FIG. 10F presents bar graph summarizing the flow data shown in panels from B to E.

The efficacy of CARIR-expressing THP-1 macrophages to phagocytose tumor cells was then investigated. To serve as the effectors, human monocytic THP-1 cells were engineered to express either CARIR-Δz or CARIR-z through lentiviral transduction, and then differentiated to macrophages by PMA treatment. MDA-MB-231 tumor cells that naturally express PD-LA were used to serve as the target cells (FIG. 11A). FIG. 11B is a histogram showing the PD-L1 expression on MDA-MB-231 cells. Representative flow plots showing % phagocytosis on MDA-MB-231 tumor cells are shown in FIG. 11C. The data are summarized in FIG. 11D.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A chimeric receptor comprising, from the N-terminus to the C-terminus, an extracellular domain of a receptor to a tumor-associated ligand, a transmembrane domain, and a CD3 intracellular domain.

2. The chimeric receptor of claim 1, wherein the extracellular domain comprises a ligand-binding domain.

3. The chimeric receptor of claim 1, wherein the receptor has a binding affinity to the tumor-associated ligand that is between 100 μM and 100 nM (EC₅₀).

4. The chimeric receptor of claim 1, wherein the receptor is selected from the group consisting of PD1, SIRPα, Siglec-10, CTLA-4, CXCR-4, CCR-2, CXCR2, CCR7, CD80, TIM-3, LAG3 and TREM2.

5. The chimeric receptor of claim 4, wherein the receptor is PD1.

6. The chimeric receptor of claim 4, wherein the extracellular domain of the receptor comprises an amino acid sequence selected from the group consisting of SEQ ID NO:2, 5, 8, 11, 18, 20, 22, 24 and 42.

7. The chimeric receptor of claim 1, further comprising a costimulatory domain.

8. The chimeric receptor of claim 7, wherein the costimulatory domain is a signaling domain of a protein selected from the group consisting of CD28, CD27, OX40, CD40, CD80, CD86, and 4-1BB.

9. (canceled)

10. An immune cell comprising the chimeric receptor of claim 1.

11. The immune cell of claim 10, wherein the immune cell is selected from the group consisting of myeloid cell, natural killer (NK) cell, T cell, tumor infiltrating lymphocyte, and natural killer T (NKT) cell.

12. The immune cell of claim 10, wherein the immune cell is p50 deficient.

13. (canceled)

14. The immune cell of claim 10, wherein the immune cell is a myeloid cell.

15. The immune cell of claim 14, wherein the immune cell is a p50 deficient immature myeloid cell.

16. The immune cell of claim 10, wherein the immune cell further comprises exogenous polynucleotide encoding a proinflammatory cytokine.

17. The immune cell of claim 16, wherein the proinflammatory cytokine is selected from the group consisting of IL-12, IFN-γ, TNF-α, and IL-1β.

18-19. (canceled)

20. A polynucleotide encoding the chimeric receptor of claim 1.

21-22. (canceled)

23. A method of treating a cancer in a patient, comprising administering to the patient the immune cell of claim 10.

24. The method of claim 23, wherein the cancer is a solid tumor, a leukemia or lymphoma.

25-26. (canceled)

27. The method of claim 23, wherein the cancer is metastatic.


SEQUENCE LISTING (43 sequences)

SEQ ID NO: 1 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 30 aa, synthetic construct
MSPSNQTDKLAAFPEDRSQPGQDCRFRVTQ

SEQ ID NO: 3 - protein, 101 aa, synthetic construct
MSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 4 - protein, 504 aa, synthetic construct
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 5 - protein, 103 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVE

SEQ ID NO: 6 - protein, 235 aa, synthetic construct
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRA

SEQ ID NO: 7 - protein, 697 aa, synthetic construct
MLLPLLLSSLLGGSQAMDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSNGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 8 - protein, 129 aa, synthetic construct
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQK

SEQ ID NO: 9 - protein, 238 aa, synthetic construct
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALE

SEQ ID NO: 10 - protein, 223 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 11 - protein, 99 aa, synthetic construct
RGIASFVCEYASPGKATEVRVTVLRQADSQVTEVCAATYMMGNELTFLDDSICTGTSSGNQVNLTIQGLRAMDTGLYICKVELMYPPPYYLGIGNGTQI

SEQ ID NO: 12 - protein, 301 aa, synthetic construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 13 - protein, 180 aa, synthetic construct
EVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 14 - protein, 525 aa, synthetic construct
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 15 - protein, 237 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTV

SEQ ID NO: 16 - protein, 353 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVS

SEQ ID NO: 17 - protein, 352 aa, synthetic construct
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 18 - protein, 38 aa, synthetic construct
MEGISIYTSDNYTEEMGSGDYDSMKEPCFREENANFNK

SEQ ID NO: 19 - protein, 360 aa, synthetic construct
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 20 - protein, 37 aa, synthetic construct
LSTSRSRFIRNTNESGEEVTTFFDYDYGAPCHKFDVK

SEQ ID NO: 21 - protein, 360 aa, synthetic construct
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 22 - protein, 47 aa, synthetic construct
EDFNMESDSFEDFWKGEDLSNYSYSSTLPPFLLDAAPCEPESLEINK

SEQ ID NO: 23 - protein, 378 aa, synthetic construct
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 24 - protein, 34 aa, synthetic construct
QDEVTDDYIGDNTTVDYTLFESLCSKKDVRNFKA

SEQ ID NO: 25 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 26 - protein, 17 aa, synthetic construct
MWLQSLLLLGTVACSIS

SEQ ID NO: 27 - protein, 49 aa, synthetic construct
KPTTTPAPRPPTPAPTIASQPLSLRPEACRPAAGGAVHTRGLDFACDIY

SEQ ID NO: 28 - protein, 21 aa, synthetic construct
IWAPLAGTCGVLLLSLVITLY

SEQ ID NO: 29 - protein, 19 aa, synthetic construct
ATNFSLLKQAGDVEENPGP

SEQ ID NO: 30 - protein, 18 aa, synthetic construct
EGRGSLLTCGDVEENPGP

SEQ ID NO: 31 - protein, 62 aa, synthetic construct
KKVAKKPTNKAPHPKQEPQEINFPDDLPGSNTAAPVQETLHGCQPVTQEDGKESRISVQERQ

SEQ ID NO: 32 - DNA, 1599 bp, synthetic construct
  source 1..1599: mol_type = other DNA
atgtgccaccagcaacttgtaataagttggttctcactggtcttccttgcgagcccgttggtagcaatctgggaactgaaaaaggacgtatatgtggtggagctcgattggtatcccgacgcacccggggagatggttgtcctcacctgtgataccccagaggaagacggtatcacttggaccttggatcagtcctccgaggtgcttggaagcggaaaaactctgacgatacaggtgaaagaattcggcgacgcgggacagtatacatgccacaaaggcggggaggtactgtcacattcactcctgctcttgcataaaaaggaggacgggatctggagcactgatattttgaaagaccaaaaagaaccaaaaaacaagaccttcctcaggtgtgaggccaagaattatagtggacgcttcacctgctggtggctgaccacaattagtactgacttgactttctcagtaaagagttccagggggagtagtgacccccaaggggtaacctgcggggccgcgactttgtcagcggaacgggtgcgaggggataataaggaatatgagtattcagtggagtgccaggaagactctgcatgtcccgcagcagaggaaagcctgccgatagaggtaatggttgacgccgtccataagctcaaatacgagaactacacaagctctttcttcatacgggacattattaagccagaccccccgaagaatcttcaacttaaaccgttgaaaaatagtcgacaagtggaagtcagttgggaatatccagacacctggagtacgccacacagctatttttccttgacattttgcgtccaggttcaggggaaatcaaagcgcgagaagaaggatcgagtctttacagacaagacgagtgcaacggtaatctgtaggaagaacgcaagcatttccgtcagagctcaggaccggtactatagcagttcatggtctgaatgggctagtgtaccttgcagtggaggtggcggcggcggctctcgaaatctgccagtcgctaccccggacccaggaatgtttccatgcctgcaccacagtcagaacctgctccgggcggtttccaacatgcttcagaaagcgcgccagacccttgaattttacccctgcacaagtgaagagatagaccatgaagatattaccaaggataaaacatcaactgtagaggcgtgtctccctctcgaactgacaaagaacgagtcttgtctcaatagtagggaaacttcattcattacaaacgggtcatgtcttgcttcaaggaagaccagcttcatgatggcactctgcttgtcttcaatctatgaggatcttaaaatgtaccaagtagagtttaagactatgaatgcgaagctcctgatggatccgaagcggcagatttttttggaccagaatatgttggcggtcattgacgaacttatgcaagctctcaatttcaattcagagacggttcctcagaaaagctccttggaagagccggacttctacaaaactaagatcaaattgtgtatcttgctccatgcattccggatacgcgccgtgaccattgatcgagtaatgtcctatttgaatgcaagctaa

SEQ ID NO: 33 - protein, 532 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - DNA, 984 bp, synthetic construct
  source 1..984: mol_type = other DNA
atgtgccaccagcaacttgtaataagttggttctcactggtcttccttgcgagcccgttggtagcaatctgggaactgaaaaaggacgtatatgtggtggagctcgattggtatcccgacgcacccggggagatggttgtcctcacctgtgataccccagaggaagacggtatcacttggaccttggatcagtcctccgaggtgcttggaagcggaaaaactctgacgatacaggtgaaagaattcggcgacgcgggacagtatacatgccacaaaggcggggaggtactgtcacattcactcctgctcttgcataaaaaggaggacgggatctggagcactgatattttgaaagaccaaaaagaaccaaaaaacaagaccttcctcaggtgtgaggccaagaattatagtggacgcttcacctgctggtggctgaccacaattagtactgacttgactttctcagtaaagagttccagggggagtagtgacccccaaggggtaacctgcggggccgcgactttgtcagcggaacgggtgcgaggggataataaggaatatgagtattcagtggagtgccaggaagactctgcatgtcccgcagcagaggaaagcctgccgatagaggtaatggttgacgccgtccataagctcaaatacgagaactacacaagctctttcttcatacgggacattattaagccagaccccccgaagaatcttcaacttaaaccgttgaaaaatagtcgacaagtggaagtcagttgggaatatccagacacctggagtacgccacacagctatttttccttgacattttgcgtccaggttcaggggaaatcaaagcgcgagaagaaggatcgagtctttacagacaagacgagtgcaacggtaatctgtaggaagaacgcaagcatttccgtcagagctcaggaccggtactatagcagttcatggtctgaatgggctagtgtaccttgcagt

SEQ ID NO: 35 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 36 - protein, 7 aa, synthetic construct
GGGGGGS

SEQ ID NO: 37 - DNA, 594 bp, synthetic construct
  source 1..594: mol_type = other DNA
cgaaatctgccagtcgctaccccggacccaggaatgtttccatgcctgcaccacagtcagaacctgctccgggcggtttccaacatgcttcagaaagcgcgccagacccttgaattttacccctgcacaagtgaagagatagaccatgaagatattaccaaggataaaacatcaactgtagaggcgtgtctccctctcgaactgacaaagaacgagtcttgtctcaatagtagggaaacttcattcattacaaacgggtcatgtcttgcttcaaggaagaccagcttcatgatggcactctgcttgtcttcaatctatgaggatcttaaaatgtaccaagtagagtttaagactatgaatgcgaagctcctgatggatccgaagcggcagatttttttggaccagaatatgttggcggtcattgacgaacttatgcaagctctcaatttcaattcagagacggttcctcagaaaagctccttggaagagccggacttctacaaaactaagatcaaattgtgtatcttgctccatgcattccggatacgcgccgtgaccattgatcgagtaatgtcctatttgaatgcaagctaa

SEQ ID NO: 38 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 39 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 41 - protein, 288 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 42 - protein, 13 aa, synthetic construct
IFDITNNLSIVIL

SEQ ID NO: 43 - protein, 105 aa, synthetic construct
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVK